(19) United States
(12) Patent Application Publication
(10) Pub. No.: US 2022/0275100 A1
(43) Pub. Date: Sep. 1, 2022

(54) ANTI-CD38 ANTIBODY, ANTIGEN-BINDING FRAGMENT THEREOF, AND PHARMACEUTICAL USE

(71) Applicants: Jiangsu Hengrui Medicine Co., Ltd., Lianyungang, Jiangsu (CN); Shanghai Hengrui Pharmaceutical Co., Ltd., Shanghai (CN)

(72) Inventors: Xin Ye, Shanghai (CN); Le Sun, Shanghai (CN); Mingjuan Song, Shanghai (CN); Beibei Fu, Shanghai (CN); Xiaohua Wang, Shanghai (CN); Lei Zhang, Shanghai (CN); Weikang Tao, Shanghai (CN)

(21) Appl. No.: 17/274,082

(22) PCT Filed: Sep. 10, 2019
(86) PCT No.: PCT/CN2019/105119

§ 371 Date: Mar. 5, 2021

(30) Foreign Application Priority Data
Sep. 11, 2018 (CN) 201811060341.2

(51) Int. Cl. C07K 16/28; G01N 33/574; A61P 35/00

(52) U.S. Cl. CPC C07K 16/2896; G01N 33/57492; A61P 35/00; A61K 2039/505

(57) ABSTRACT

The present application provides an anti-CD38 antibody, an antigen-binding fragment thereof, and pharmaceutical use. Specifically, the present application provides a murine-derived antibody, a chimeric antibody or a humanized antibody comprising a CDR region of the anti-CD38 antibdoy, a pharmaceutical composition comprising the anti-CD38 antibody or the antigen-binding fragment thereof, and an application thereof as a drug. In particular, the present application provides an application of a humanized anti-CD38 antibody in preparing a drug for treating a CD38 positive disease or disorder.

### CROSS-REFERENCE TO RELATED APPLICATIONS

This application is a U.S. National Phase of International PCT Application No. PCT/CN2019/105119 filed Sep. 10, 2019, which claims priority to Chinese Patent Application Serial No. 201811060341.2 filed Sep. 11, 2018, the contents of each application are incorporated herein by reference in their entirety.

### SEQUENCE LISTING

This application incorporates by reference the material in the ASCII text file titled Amended Sequence Listing.txt, which was created on Oct. 19, 2021 and is 81 KB.

### FIELD OF THE INVENTION

The present disclosure belongs to the field of biotechnology. More specifically, the present disclosure relates to therapeutic uses of anti-CD38 antibodies and compositions thereof and methods for producing the antibody molecules.

### BACKGROUND OF THE INVENTION

The descriptions herein only provide background information about the present disclosure, and do not necessarily constitute prior art.

Multiple myeloma (MM) is a malignant plasma cell disease, in which tumor cells are derived from plasma cells in bone marrow, whereas the plasma cells are cells at the final functional stage developed from B lymphocytes. Therefore, multiple myeloma can also be classified as B lymphocyte lymphoma.

Multiple myeloma is characterized by abnormal proliferation of bone marrow plasma cells, accompanied by excessive production of monoclonal immunoglobulin or light chain (M protein); whereas non-secretory MM that does not produce M protein in few patients. Multiple myeloma is often accompanied by multiple osteolytic damage, hypercalcemia, anemia, and kidney damage. Various bacterial infections are easily to be found due to the inhibition of the production of normal immunoglobulin.

The domestic morbidity in China is estimated to be 2 to 3 per 100,000 persons, and the male to female ratio is 1.6:1. Most patients are older than 40 years old, and most are older than 60 years old. At present, there are estimated to be about 70,000 patients in China, and there are about 80,000 new patients worldwide each year. Multiple myeloma accounts for about 10% of various hematopoietic tumors. The number of patients has a tendency to further increase with the increased aging.

At present, multiple myeloma is an incurable hematological malignant tumor. The currently main treatments include: bone marrow transplantation, multiple small molecule chemotherapies, especially protease inhibitors represented by carfilzomib, and immunomodulators represented by lenalidomide, they greatly prolong the survival of MM patients. However, the disease almost always recurs in the end, and the average survival after relapse is only about 9 months. Daratumumab (Dara) available from Janssen Company alone showed good results in patients with relapsed or refractory multiple myeloma (RRMM), so FDA regarded it as a breakthrough, and Daratumumab was approved for marketing in November 2015 for the treatment of RRMM patients. Currently, several clinical trials with Dara in combination with small molecules have been carried out. Dara in combination with small molecule drugs can greatly improve the clinical efficacy.

CD38 is a type II transmembrane multifunctional protein with an extracellular domain of 256 amino acids. In one aspect, CD38 has been indirectly demonstrated to play a role in lateral signaling through the ligand CD31, which can cause cell adhesion and play a role in lymphocyte activation and B cell differentiation. However, there is no direct biochemical evidence for this function. On the other hand, CD38 has functions as cyclase and hydrolase, and it can not only promote the conversion of NAD (Nicotinamide Adenine Dinucleotide) into cADPR (cyclic Adenosine Diphosphate Ribose) by the action of cyclase, but also promote the conversion of cADPR into ADPR (Adenosine Diphosphate Ribose) by the action of cADPR hydrolase. CD38 can regulate Ca²⁺ flow, and it can also promote the production of adenosine in cells to suppress immunity. Changes in Ca²⁺ flow may affect the secretion of insulin. The mice with CD38 gene being knocked out showed normal survival, but exhibited various symptoms such as decreased humoral immune response, decreased insulin, and heart/pulmonary muscle defects. CD38 in humans is expressed in hematopoietic cells, immune cells, and various normal tissues such as brain, pancreas, kidney, muscle, and the like. It was found highly expressed in prostate and thymus on RNA level, whereas the expression level was significantly increased in a variety of hematologic tumor cells, especially in multiple myeloma cells. The clinical data of Daratumumab also verified the efficacy and safety of CD38 antibody in the field of multiple myeloma, suggesting the potential value of CD38 target development. Various studies in literatures have shown that the mechanism underlying the treatment of MM with anti-CD38 antibodies mainly includes:

1) ADCC, CDC, ADCP, mechanism mainly related to epitopes and IgG1-Fc;

2) direct killing effect caused by apoptosis;

3) affecting tumor cell survival by inhibiting CD38 enzyme activity;

4) potential clearance for Treg cells expressing CD38.

The CD38 target has become a hot spot in the treatment of multiple myeloma. Another important CD38-targeting antibody is Isartuximab available from Sanofi, which has also been used in a number of clinical trials for single use and in combination with small molecule drugs. From the existing data, Isartuximab has shown the same efficacy and safety as Daratumumab. At present, antibodies such as MOR202 (Morphasys) and TAK-079 (Takeda) have entered the clinic, and there are also CD38/CD3 bispecific antibodies and CD38-CAR-T being in the preclinical research phase. Other patents related to antibodies against CD38 as target can be found in, for example, WO2006099875, WO2007042309, WO2008047242, WO2012092612, WO2016164656, WO2017149122, etc.

Currently, there is still an urgent need to continue to develop antibodies with high selectivity, high affinity and favorable efficacy.

### SUMMARY OF THE INVENTION

The present disclosure provides a series of CD38 antibodies with higher affinity, better anti-tumor activity in vivo, and favorable metabolic activity in vivo. Specifically, the present disclosure provides a monoclonal antibody or an antigen-binding fragment that specifically binds to human CD38.

In some embodiments, the present disclosure provides an anti-CD38 antibody or an antigen-binding fragment thereof, the antibody specifically binding to human CD38, and the antibody or the antigen-binding fragment thereof comprises CDRs as shown below:

(i) heavy chain HCDR1, HCDR2, HCDR3 as shown in SEQ ID Nos: 9, 10 and 11 respectively, or HCDR variants having 3, 2 or 1 amino acid(s) difference(s) when compared with HCDR1, HCDR2, and HCDR3 as shown in SEQ ID Nos: 9, 10, and 11 respectively; and light chain LCDR1, LCDR2, LCDR3 as shown in amino acid sequence SEQ ID Nos: 12, 13 and 14 respectively, or LCDR variants having 3, 2 or 1 amino acid(s) difference(s) when compared with LCDR1, LCDR2, LCDR3 as shown in SEQ ID Nos: 12, 13 and 14 respectively; or

(ii) heavy chain HCDR1, HCDR2, HCDR3 as shown in SEQ ID Nos: 15, 16 and 17 respectively, or HCDR variants having 3, 2 or 1 amino acid(s) difference(s) when compared with HCDR1, HCDR2, and HCDR3 as shown in SEQ ID Nos: 15, 16, and 17 respectively; and light chain LCDR1, LCDR2, LCDR3 as shown in amino acid sequence SEQ ID Nos: 18, 19 and 20 respectively, or LCDR variants having 3, 2 or 1 amino acid(s) difference(s) when compared with LCDR1, LCDR2, LCDR3 as shown in SEQ ID Nos: 18, 19 and 20 respectively; or

(iii) heavy chain HCDR1, HCDR2, HCDR3 as shown in SEQ ID Nos: 15, 21 and 17 respectively, or HCDR variants having 3, 2 or 1 amino acid(s) difference(s) when compared with HCDR1, HCDR2, and HCDR3 as shown in SEQ ID Nos: 15, 21, and 17 respectively; and light chain LCDR1, LCDR2, LCDR3 as shown in amino acid sequence SEQ ID Nos: 22, 19 and 23 respectively, or LCDR variants having 3, 2 or 1 amino acid(s) difference(s) when compared with LCDR1, LCDR2, LCDR3 as shown in SEQ ID Nos: 22, 19 and 23 respectively.

In some embodiments, the CDR variants having 3, 2 or 1 amino acid(s) difference(s) of the CDR (including 3 heavy chain CDRs and 3 light chain CDRs) of the anti-CD38 antibody or the antigen-binding fragment are CDR variants with 3, 2 or 1 amino acid difference(s) obtained by screening by affinity maturation methods.

In some embodiments, the affinity (KD) of the anti-CD38 antibody or the antigen-binding fragment thereof to human CD38 is less than 10⁻⁸ M, less than 10⁻⁹M, less than 10⁻¹⁰ M, or less than 10⁻¹¹ M.

In some embodiments, the anti-CD38 antibody is a murine, a chimeric or a humanized antibody, preferable is a humanized antibody.

In some embodiments, the antibody is a murine antibody or a chimeric antibody, and the amino acid sequence of the heavy chain variable region of the antibody is as shown in SEQ ID Nos: 3, 5, 7, or having at least 95%, 96%, 97%, 98%, 99% or 100% sequence identity to SEQ ID Nos: 3, 5, 7; and/or the amino acid sequence of the light chain variable region of the antibody is as shown in SEQ ID Nos: 4, 6, 8, or having at least 95%, 96%, 97%, 98%, 99% or 100% sequence identity to SEQ ID Nos: 4, 6, 8.

In some embodiments, the anti-CD38 antibody or the antigen-binding fragment thereof comprises a heavy chain variable region and a light chain variable region as shown below:

(a):

a heavy chain variable region, the amino acid sequence thereof is as shown in SEQ ID No: 3 or has at least 95%, 96%, 97%, 98%, 99%, or 100% sequence identity to SEQ ID No: 3, and

a light chain variable region, the amino acid sequence thereof is as shown in SEQ ID No: 4 or has at least 95%, 96%, 97%, 98%, 99% or 100% sequence identity to SEQ ID No: 4;

(b):

a heavy chain variable region, the amino acid sequence thereof is as shown in SEQ ID No: 5 or has at least 95%, 96%, 97%, 98%, 99%, or 100% sequence identity to SEQ ID No: 5, and

a light chain variable region, the amino acid sequence thereof is as shown in SEQ ID No: 6 or has at least 95%, 96%, 97%, 98%, 99% or 100% sequence identity to SEQ ID No: 6;

(c):

a heavy chain variable region, the amino acid sequence thereof is as shown in SEQ ID No: 7 or has at least 95%, 96%, 97%, 98%, 99%, or 100% sequence identity to SEQ ID No: 7, and

a light chain variable region, the amino acid sequence thereof is as shown in SEQ ID No: 8 or has at least 95%, 96%, 97%, 98%, 99% or 100% sequence identity to SEQ ID No: 8.

In some embodiments, the antibody is a humanized antibody, and the humanized antibody comprises framework (FR) regions or framework region variants derived from human germline, and the framework region variants have up to 10 (for example, 10, 9, 8, 7, 6, 5, 4, 3, 2, or 1) amino acid back-mutations on light chain framework regions and/or heavy chain framework regions of the human antibody, respectively. In some embodiments, the humanized antibody comprises any one selected from the following (d) to (f):

(d) a heavy chain variable region, wherein a heavy chain variable region comprising heavy chain HCDR1, HCDR2, HCDR3 and heavy chain framework region(s), wherein:

the amino acid sequence of the HCDR1 is as shown in SEQ ID No: 9 or has 3, 2 or 1 amino acid(s) difference(s) when compared with SEQ ID No: 9,

the amino acid sequence of the HCDR2 is as shown in SEQ ID No: 10 or has 3, 2 or 1 amino acid(s) difference(s) when compared with the sequence of SEQ ID No: 10,

the amino acid sequence of the HCDR3 is as shown in SEQ ID No: 11 or has 3, 2 or 1 amino acid(s) difference(s) when compared with the sequence of SEQ ID No: 11,

the heavy chain framework region(s) has/have one or more back-mutations selected from group consisting of 2F, 38K, 44S, 48I, 67A, 66K, 69L, 71V and 73Q; and/or

a light chain variable region, wherein a light chain variable region comprising light chain LCDR1, LCDR2, LCDR3 and light chain framework region(s), wherein:

the amino acid sequence of the LCDR1 is as shown in SEQ ID No: 12 or has 3, 2 or 1 amino acid(s) difference(s) when compared with SEQ ID No: 12,

the amino acid sequence of the LCDR2 is as shown in SEQ ID No: 13 or has 3, 2 or 1 amino acid(s) difference(s) when compared with the sequence of SEQ ID No: 13,

the amino acid sequence of the LCDR3 is as shown in SEQ ID No: 14 or has 3, 2 or 1 amino acid(s) difference(s) when compared with the sequence of SEQ ID No: 14,

the light chain framework region(s) has/have one or more back-mutations selected from group consisting of 2F, 43S, 49K and 87F;

(e) a heavy chain variable region, wherein a heavy chain variable region comprising heavy chain HCDR1, HCDR2, HCDR3 and heavy chain framework region(s), wherein:

the amino acid sequence of the HCDR1 is as shown in SEQ ID No: 15 or has 3, 2 or 1 amino acid(s) difference(s) when compared with SEQ ID No: 15,

the amino acid sequence of the HCDR2 is as shown in SEQ ID No: 16 or has 3, 2 or 1 amino acid(s) difference(s) when compared with the sequence of SEQ ID No: 16,

the amino acid sequence of the HCDR3 is as shown in SEQ ID No: 17 or has 3, 2 or 1 amino acid(s) difference(s) when compared with the sequence of SEQ ID No: 17,

the heavy chain framework region(s) has/have one or more back-mutations selected from group consisting of 79F, 82A T, 91S and 76S; and/or

a light chain variable region, wherein a light chain variable region comprising light chain LCDR1, LCDR2, LCDR3 and light chain framework region(s), wherein:

the amino acid sequence of the LCDR1 is as shown in SEQ ID No: 18 or has 3, 2 or 1 amino acid(s) difference(s) when compared with SEQ ID No: 18,

the amino acid sequence of the LCDR2 is as shown in SEQ ID No: 19 or has 3, 2 or 1 amino acid(s) difference(s) when compared with the sequence of SEQ ID No: 19,

the amino acid sequence of the LCDR3 is as shown in SEQ ID No: 20 or has 3, 2 or 1 amino acid(s) difference(s) when compared with the sequence of SEQ ID No: 20,

the light chain framework region(s) has/have one or more back-mutations selected from group consisting of 58I, 68R and 85T;

(f) a heavy chain variable region, wherein a heavy chain variable region comprising heavy chain HCDR1, HCDR2, HCDR3 and heavy chain framework region(s), wherein:

the amino acid sequence of the HCDR1 is as shown in SEQ ID No: 15 or has 3, 2 or 1 amino acid(s) difference(s) when compared with SEQ ID No: 15,

the amino acid sequence of the HCDR2 is as shown in SEQ ID No: 21 or has 3, 2 or 1 amino acid(s) difference(s) when compared with the sequence of SEQ ID No: 21,

the amino acid sequence of the HCDR3 is as shown in SEQ ID No: 17 or has 3, 2 or 1 amino acid(s) difference(s) when compared with the sequence of SEQ ID No: 17,

the heavy chain framework region(s) has/have one or more back-mutations selected from group consisting of 48I, 77T and 82A T; and/or

a light chain variable region, wherein a light chain variable region comprising light chain LCDR1, LCDR2, LCDR3 and light chain framework region(s), wherein:

The amino acid sequence of the LCDR1 is as shown in SEQ ID No: 22 or has 3, 2 or 1 amino acid(s) difference(s) when compared with SEQ ID No: 22,

the amino acid sequence of the LCDR2 is as shown in SEQ ID No: 19 or has 3, 2 or 1 amino acid(s) difference(s) when compared with the sequence of SEQ ID No: 19,

the amino acid sequence of the LCDR3 is as shown in SEQ ID No: 23 or has 3, 2 or 1 amino acid(s) difference(s) when compared with the sequence of SEQ ID No: 23,

the light chain framework region(s) has/have one or more back-mutations selected from group consisting of 4L, 9A, 22S, 58I, 60A and 68R;

wherein, the back-mutation sites described above are numbered according to Kabat Numbering Criteria, and the mutation described above such as “2F” refers to that the amino acid at position 2 (numbered according to the Kabat Numbering Criteria) has been back-mutated to phenylalanine (Phe or F), “82A T” refers to that the amino acid at position 82A (numbered according to the Kabat Numbering Criteria) has been back-mutated to threonine (Thr or T).

In some embodiments, the antibody heavy chain FR regions are selected from the combination of human germline IGHV1-3*01 and hJH4.1 or have at least 95%, 96%, 97%, 98%, 99% or 100% sequence identity thereto.

In some embodiments, the light chain FR regions are selected from the combination of human germline IGKV3-11*01 and hJK4.1 or have at least 95%, 96%, 97%, 98%, 99% or 100% sequence identity thereto.

In some embodiments, the heavy chain FR regions are selected from the combination of human germline IGHV3-7*01 and hJH6.1 FR4 or have at least 95%, 96%, 97%, 98%, 99% or 100% sequence identity thereto.

In some embodiments, the light chain FR regions are selected from the combination of human germline IGKV4-1*01 and hJK4.1 or have at least 95%, 96%, 97%, 98%, 99% or 100% sequence identity thereto.

In some embodiments, the humanized antibody comprises a heavy chain variable region as shown in SEQ ID Nos: 24, 32, or 37 or a variant thereof; wherein the variant has 1-10 (for example, 10, 9, 8, 7, 6, 5, 4, 3, 2, or 1) amino acid mutations on the framework regions of the above heavy chain variable region. In some embodiments, the amino acid mutation is selected from any of the following:

(g) one or more amino acid back-mutation(s) selected from the group consisting of 2F, 38K, 44S, 48I, 67A, 66K, 69L, 71V and 73Q on the framework regions of the heavy chain variable region as shown in SEQ ID No: 24;

(h) one or more amino acid back-mutation(s) selected from the the group consisting of 79F and 91S on the framework regions of the heavy chain variable region as shown in SEQ ID No: 32;

(i) back-mutation of 48I on the framework regions of the heavy chain variable region as shown in SEQ ID No: 37.

In some embodiments, the humanized antibody comprises:

the heavy chain variable region as shown in SEQ ID Nos: 26, 27, 28, 29, 34 or 39, or the heavy chain variable region having at least 95%, 96%, 97%, 98%, 99% or 100% sequence identity to SEQ ID Nos: 26, 27, 28, 29, 34 or 39.

In some embodiments, the humanized antibody comprises the light chain variable region as shown in SEQ ID Nos: 25, 33, or 38 or a variant thereof; the variant has 1-10 (e.g., 10, 9, 8, 7, 6, 5, 4, 3, 2 or 1) amino acid mutation(s) in the light chain variable region as shown in any of SEQ ID Nos: 25, 33, or 38.

In some embodiments, the amino acid mutation is selected from any one of the following (j) to (1):

(j) one or more amino acid back-mutation(s) selected from the the group consisting of 2F, 43S, 49K and 87F on the framework regions of the light chain variable region as shown in SEQ ID No: 25;

(k) one or more amino acid back-mutation(s) selected from the the group consisting of 58I, 68R and 85T on the framework regions of the light chain variable region as shown in SEQ ID No: 33;

(l) one or more amino acid back-mutation(s) selected from the the group consisting of 4L, 9A, 22S, 58I, 60A and 68R on the framework regions of the light chain variable region as shown in SEQ ID No: 38;

wherein, the back-mutation sites are numbered according to Kabat numbering system.

In some embodiments, the humanized antibody comprises a light chain variable region as shown in SEQ ID Nos: 30, 31, 35, 36, 40, 41 or 42, or having at least 95%, 96%, 97%, 98%, 99%, or 100% sequence identity to SEQ ID Nos: 30, 31, 35, 36, 40, 41, or 42.

In some embodiments, the humanized antibody comprises:

(m) a heavy chain variable region as shown in SEQ ID Nos: 24, 26, 27, 28 or 29, and a light chain variable region as shown in SEQ ID No:25, 30 or 31; or

(n) a heavy chain variable region as shown in SEQ ID Nos: 32 or 34, and a light chain variable region as shown in SEQ ID No:33, 35 or 36; or

(o) a heavy chain variable region as shown in SEQ ID Nos: 37 or 39, and a light chain variable region as shown in SEQ ID No:38, 40, 41 or 42;

In some embodiments, the humanized antibody comprises:

(p) a heavy chain variable region as shown in SEQ ID No:26, and a light chain variable region as shown in SEQ ID No:30; or

(q) a heavy chain variable region as shown in SEQ ID No:32, and a light chain variable region as shown in SEQ ID No:33; or

(r) a heavy chain variable region as shown in SEQ ID No:37, and a light chain variable region as shown in SEQ ID No:38.

In some embodiments, the antibody further comprises a constant region; preferably, the antibody is a chimeric antibody or humanized antibody, the heavy chain constant region of which is derived from human antibody IgG1, IgG2, IgG3 or IgG4, or conventional variants of IgG1, IgG2, IgG3 or IgG4; the light chain constant region of which is derived from human antibody kappa, lambda chains or conventional variants thereof. In some specific embodiments, the amino acid sequence of the heavy chain constant region is as shown in SEQ ID Nos: 43 or 44, or has at least 95%, 96%, 97%, 98%, 99% or 100% sequence identity thereto; the amino acid sequence of the light chain constant region is as shown in SEQ ID Nos: 45 or has at least 95%, 96%, 97%, 98%, 99%, or 100% sequence identity thereto.

In some embodiments, the heavy chain is as shown in SEQ ID Nos: 46, 48, 49, 51, 52, or 54, or has at least 85%, 86%, 87%, 88%, 89%, 90%, 91%, 92%, 93%, 94%, 95%, 96%, 97%, 98%, 99%, or 100% sequence identity thereto; and/or

the light chain is as shown in SEQ ID Nos: 47, 50 or 53, or has at least 85%, 86%, 87%, 88%, 89%, 90%, 91%, 92%, 93%, 94%, 95%, 96%, 97%, 98%, 99% or 100% sequence identity thereto.

In some embodiments, the anti-CD38 antibody comprises:

(i) a heavy chain as shown in SEQ ID Nos: 46 or 48, and a light chain as shown in SEQ ID No: 47; or

(ii) a heavy chain as shown in SEQ ID Nos: 49 or 51, and a light chain as shown in SEQ ID No: 50; or

(iii) a heavy chain as shown in SEQ ID Nos: 52 or 54, and a light chain as shown in SEQ ID No: 53.

In some specific embodiments, the anti-CD38 antibody comprises:

(iv) a heavy chain as shown in SEQ ID No: 48 and a light chain as shown in SEQ ID No: 47; or

(v) a heavy chain as shown in SEQ ID No: 51 and a light chain as shown in SEQ ID No: 50; or

(vi) a heavy chain as shown in SEQ ID No: 54 and a light chain as shown in SEQ ID No: 53.

In some embodiments, the anti-CD38 antibody or the antigen-binding fragment thereof has enhanced ADCC activity, which is achieved by modifying the affinity of the Fc region of the antibody or the antigen-binding fragment thereof to FcγIIIa. For example, mutations F243L, R292P, Y300L and combinations thereof mentioned in Patent WO2008140603 of MacroGenics, mutations S239D, I332E or combination thereof on the IgG1 Fc region mentioned in Patent US20080260731 of Xencor, and other mutations capable of enhancing ADCC function disclosed in the art.

In some embodiments, the antigen-binding fragment is selected from the group consisting of Fab, Fab′, F(ab′)2, scFv, diabody, and dsFv.

The present disclosure also provides an anti-CD38 antibody competing with the antibody or the antigen-binding fragment thereof described above for binding to human CD38, or competing with the antibody or the antigen-binding fragment thereof described above for binding to the same epitope of CD38 antigen.

The present disclosure also provides a pharmaceutical composition, comprising a therapeutically effective amount of the anti-CD38 antibody or the antigen-binding fragment thereof described above, and one or more pharmaceutically acceptable carriers, diluents, buffers, or excipients. Preferably, the pharmaceutical composition may contain 0.01 to 99% by weight of the anti-CD38 antibody or the antigen-binding fragment thereof in unit dosage; or the pharmaceutical composition may contain, preferably 0.1-2000 mg, more preferably 1-1000 mg of the antibody or the antigen-binding fragment thereof in unit dosage.

The present disclosure also provides an isolated nucleic acid molecule, which encodes the anti-CD38 antibody or the antigen-binding fragment thereof described above.

The present disclosure also provides a vector, which comprises the nucleic acid molecule described above.

The present disclosure also provides a host cell transformed (or transduced or transfected) with the vector described above. Also discloses a host cell, which contains the vector described above. The host cell is selected from prokaryotic cell and eukaryotic cell. In some embodiments, the host cell does not include any human cell capable of developing into a complete individual, such as human embryonic stem cell, fertilized egg, and germ cell; preferably, the host cell is a eukaryotic cell, more preferably a mammalian cell, wherein the mammalian cell includes but not limited to CHO, 293, NSO, and a mammalian cell in which gene editing is performed to change the glycosylation modification of the antibody or antigen-binding fragment thereof, thereby modifying the ADCC function of the antibody or antigen-binding fragments thereof, for example, knocking out genes such as FUT8 or GnT-III; in some embodiments, the mammalian cell do not include human cell.

The present disclosure also provides a method for preparing the anti-CD38 antibody or the antigen-binding fragments thereof described above, the method comprises the steps of cultivating the host cells described above, and then recovering the anti-CD38 antibody or the antigen-binding fragments thereof; optionally including the step of purifying the anti-CD38 antibody or the antigen-binding fragment thereof.

The present disclosure also provides a method for detecting or measuring human CD38, comprising contacting the anti-CD38 antibody or the antigen-binding fragment thereof described above with a sample to be tested.

The present disclosure also provides a reagent for detecting or measuring human CD38, the reagent comprises the anti-CD38 antibody or the antigen-binding fragment thereof described above.

The present disclosure also provides a diagnostic agent for diseases related to human CD38, the diagnostic agent comprises the anti-CD38 antibody or the antigen-binding fragment thereof described above.

The present disclosure also provides a method for diagnosing diseases related to human CD38, comprising detecting or measuring human CD38 or CD38 positive cells using the anti-CD38 antibody or the antigen-binding fragment thereof described above.

The present disclosure also provides the use of the anti-CD38 antibody or the antigen-binding fragment thereof described above in the preparation of a diagnostic agent for diseases related to human CD38.

The present disclosure also provides a method for treating or preventing a disease, comprising administering to a subject a therapeutically effective amount or a prophylactically effective amount of the anti-CD38 antibody or the antigen-binding fragment thereof described above, or a pharmaceutical composition comprising the same, or the nucleic acid molecular described above.

In some embodiments, the disease or disorder is tumor or immune disease.

In some embodiments, the disease or disorder is CD38 positive disease or disorder.

In some embodiments, the disease or disorder described above is tumor.

In some embodiments, the tumor described above is selected from the group consisting of leukemia, B cell lymphoma, plasma cell malignant tumor, T/NK cell lymphoma and myeloma. In some embodiments, the leukemia is selected from the group consisting of acute lymphocytic leukemia, acute lymphoblastic leukemia, acute promyelocytic leukemia, chronic lymphocytic leukemia, acute and chronic myeloid leukemia. In some embodiments, the myeloma is selected from the group consisting of multiple myeloma, anterior medullary tumor, light chain amyloidosis, and the like. In some embodiments, the lymphoma is non-Hodgkin's lymphoma or Hodgkin's lymphoma. In some embodiments, the tumor is B cell lymphoma/leukemia, for example selected from the group consisting of: mature B cell tumor, precursor B cell lymphoblastic leukemia/lymphoma, B cell non-Hodgkin's lymphoma and B cell Hodgkin's lymphoma. In some embodiments, the tumor is selected from the group consisting of B cell chronic lymphocytic leukemia (CLL), small lymphocytic leukemia (SLL), B cell acute lymphocytic leukemia, B cell prelymphocytic leukemia, lymphoplasmacytoid lymphoma, mantle cell lymphoma (MCL), low-grade/intermediate/high-grade follicular lymphoma (FL), cutaneous follicular central lymphoma, marginal zone B cell lymphoma (including MALT type, lymph node MZBL type, spleen MZBL type), hairy cell leukemia, diffuse large B cell lymphoma, Burkitt lymphoma, plasma cell tumor/plasma cell myeloma, plasma cell leukemia, post-transplant lymphoproliferative disease, Waldenstrom macroglobulinemia, plasma cell leukemia, anaplastic large cell lymphoma (ALCL) and hairy cell lymphoma.

In some embodiments, the tumor is B cell lymphoma or multiple myeloma.

In some embodiments, the tumor is multiple myeloma.

In some embodiments, the disease or disorder described above is an immune disease, such as an immune disease involving B cells, plasma cells, monocytes, and T cells that express CD38.

In some embodiments, the immune disease includes but not limited to: rheumatoid arthritis, psoriasis, ankylosing spondylitis, joint psoriasis, dermatitis, systemic scleroderma and sclerosis, inflammatory bowel disease (IBD), Crohn's disease, ulcerative Colitis, respiratory distress syndrome, meningitis, encephalitis, gastritis, uveitis, glomerulonephritis, eczema, asthma, arteriosclerosis, leukocyte adhesion deficiency, Raynaud syndrome, Sjogren syndrome, juvenile diabetes, Reiter disease, Behcet disease, immune complex nephritis, IgA nephropathy, IgM polyneuropathy, immune-mediated thrombocytopenia symptom (e.g. acute idiopathic thrombocytopenic purpura, chronic idiopathic thrombocytopenic purpura), hemolytic anemia, myasthenia gravis, lupus nephritis, systemic lupus erythematosus, rheumatoid arthritis (RA), atopic dermatitis, pemphigus, Graves disease, Hashimoto's thyroiditis, Wegener's granulomatosis, Omenn syndrome, chronic renal failure, acute infectious mononucleosis, multiple sclerosis, HIV and herpes virus-related diseases, severe acute respiratory syndrome and chorioretinitis, graft versus host disease, and immune disease caused by virus infection (such as disease caused or mediated by B cells infected with Ebola virus (EBV)).

In some embodiments, the immune disease is selected from the group consisting of: rheumatoid arthritis, systemic lupus erythematosus, asthma, inflammatory bowel disease, multiple sclerosis, Crohn's disease, gastritis, Hashimoto's thyroiditis, ankylosing spondylitis and graft versus host disease. In some embodiments, the disease or disorder is rheumatoid arthritis.

The present disclosure further provides the use of the anti-CD38 antibody or the antigen-binding fragment thereof described above or the pharmaceutical composition or the nucleic acid molecule described above in the preparation of a medicament for the treatment or prevention of diseases or disorders.

In some embodiments, the disease or disorder is tumor or immune disease.

In some embodiments, the disease or disorder is CD38 positive disease or disorder.

In some embodiments, the disease or disorder described above may be tumor, for example, the disease is characterized by the presence of CD38-expressing tumor cells.

In some embodiments, the tumor described above is selected from the group consisting of leukemia, B cell lymphoma, plasma cell malignant tumor, T/NK cell lymphoma and myeloma.

In some embodiments, the leukemia is selected from the group consisting of acute lymphocytic leukemia, acute lymphoblastic leukemia, acute promyelocytic leukemia, chronic lymphocytic leukemia, acute and chronic myeloid leukemia.

In some embodiments, the myeloma is selected from the group consisting of multiple myeloma, anterior medullary tumor, and light chain amyloidosis.

In some embodiments, the lymphoma is non-Hodgkin's lymphoma or Hodgkin's lymphoma.

In some embodiments, the tumor described above is B cell lymphoma/leukemia, for example is selected from mature B cell tumors or precursor B cell lymphoblastic leukemia/lymphoma, or selected from B cell non-Hodgkin's lymphoma or B cell Hodgkin's lymphoma.

In some embodiments, the tumor described above is selected from the group consisting of: B cell chronic lymphocytic leukemia (CLL), small lymphocytic leukemia (SLL), B cell acute lymphocytic leukemia, B cell prelymphocytic leukemia, lymphoplasmacytoid lymphoma, mantle cell lymphoma (MCL), follicular lymphoma (including low-grade, intermediate or high-grade FL), cutaneous follicular central lymphoma, marginal zone B cell lymphoma (including MALT type, lymph node MZBL type, spleen MZBL type), hairy cell leukemia, diffuse large B cell lymphoma, Burkitt lymphoma, plasma cell tumor, plasma cell myeloma, plasma cell leukemia, post-transplant lymphoproliferative disease, Waldenstrom macroglobulinemia, plasma cell leukemia, anaplastic large cell lymphoma (ALCL) and hairy cell lymphoma.

In some embodiments, the tumor is B cell lymphoma or multiple myeloma.

In some embodiments, the tumor is multiple myeloma.

In some embodiments, the disease or disorder is an immune disease, such as an immune disease involving B cells, plasma cells, monocytes, and T cells that express CD38.

In some embodiments, the immune disease may be selected from the group consisting of rheumatoid arthritis, psoriasis, ankylosing spondylitis, joint psoriasis, dermatitis, systemic scleroderma and sclerosis, inflammatory bowel disease (IBD), Crohn's disease, ulcerative colitis, respiratory distress syndrome, meningitis, encephalitis, gastritis, uveitis, glomerulonephritis, eczema, asthma, arteriosclerosis, leukocyte adhesion deficiency, Raynaud syndrome, Sjogren syndrome, juvenile diabetes, Reiter disease, Behcet disease, immune complex nephritis, IgA nephropathy, IgM polyneuropathy, immune-mediated thrombocytopenia symptom (e.g. acute idiopathic thrombocytopenic purpura, chronic idiopathic thrombocytopenic purpura), hemolytic anemia, myasthenia gravis, lupus nephritis, systemic lupus erythematosus, rheumatoid arthritis (RA), atopic dermatitis, pemphigus, Graves disease, Hashimoto's thyroiditis, Wegener's granulomatosis, Omenn syndrome, chronic renal failure, acute infectious mononucleosis, multiple sclerosis, HIV and herpes virus-related diseases, severe acute respiratory syndrome, chorioretinitis, graft versus host disease, and immune disease caused by virus infection (such as disease caused or mediated by B cells infected with Ebola virus (EBV)). In some embodiments, the immune disease is selected from the group consisting of: rheumatoid arthritis, systemic lupus erythematosus, asthma, inflammatory bowel disease, multiple sclerosis, Crohn's disease, gastritis, Hashimoto's thyroiditis, ankylosing spondylitis and graft versus host disease. In some embodiments, the immune disease or disorder is rheumatoid arthritis.

The present disclosure further provides an anti-CD38 antibody or an antigen-binding fragment thereof, or a pharmaceutical composition, or a nucleic acid molecule, for use in the treatment or prevention of the diseases or disorders described above.

In some embodiments, the disease or disorder is tumor or immune disease; in some embodiments, the disease or disorder is CD38 positive disease or disorder.

In some embodiments, the disease or disorder described above is tumor. For example, the disease is characterized by the presence of CD38-expressing tumor cells. In some embodiments, the tumor is selected from the group consisting of leukemia, B cell lymphoma, plasma cell malignant tumor, T/NK cell lymphoma and myeloma. In some embodiments, the leukemia is selected from the group consisting of acute lymphocytic leukemia, acute lymphoblastic leukemia, acute promyelocytic leukemia, chronic lymphocytic leukemia, acute and chronic myeloid leukemia. In some embodiments, the myeloma is selected from the group consisting of multiple myeloma, anterior medullary tumor, and light chain amyloidosis. In some embodiments, the lymphoma is non-Hodgkin's lymphoma or Hodgkin's lymphoma. In some embodiments, the tumor is B cell lymphoma/leukemia, for example selected from the group consisting of: mature B cell tumor, precursor B cell lymphoblastic leukemia/lymphoma, B cell non-Hodgkin's lymphoma and B cell Hodgkin's lymphoma. In some embodiments, the tumor is selected from the group consisting of: B cell chronic lymphocytic leukemia (CLL), small lymphocytic leukemia (SLL), B cell acute lymphocytic leukemia, B cell prelymphocytic leukemia, lymphoplasmacytoid lymphoma, mantle cell lymphoma (MCL), follicular lymphoma (including low-grade, intermediate or high-grade FL), cutaneous follicular central lymphoma, marginal zone B cell lymphoma (including MALT type, lymph node MZBL type, spleen MZBL type), hairy cell leukemia, diffuse large B cell lymphoma, Burkitt lymphoma, plasma cell tumor, plasma cell myeloma, plasma cell leukemia, post-transplant lymphoproliferative disease, Waldenstrom macroglobulinemia, plasma cell leukemia, anaplastic large cell lymphoma (ALCL) and hairy cell lymphoma. In some embodiments, the tumor is B cell lymphoma or multiple myeloma. In some embodiments, the tumor is multiple myeloma.

In some embodiments, the disease or disorder described above is immune disease, for example, immune disease involving B cells, plasma cells, monocytes, and T cells that express CD38.

In some embodiments, the immune disease may be selected from the group consisting of rheumatoid arthritis, psoriasis, joint psoriasis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, arteriosclerosis, leukocyte adhesion deficiency, multiple sclerosis, Raynaud syndrome, Sjogren syndrome, juvenile diabetes, Reiter disease, Behcet disease, immune complex nephritis, IgA nephropathy, IgM polyneuropathy, immune-mediated thrombocytopenia symptom (e.g. acute idiopathic thrombocytopenic purpura, chronic idiopathic thrombocytopenic purpura), hemolytic anemia, myasthenia gravis, lupus nephritis, systemic lupus erythematosus, rheumatoid arthritis (RA), atopic dermatitis, pemphigus, Graves disease, Hashimoto's thyroiditis, Wegener's granulomatosis, Omenn syndrome, chronic renal failure, acute infectious mononucleosis, multiple sclerosis, HIV and herpes virus-related diseases, severe acute respiratory syndrome, chorioretinitis, and immune disease caused by virus infection (such as disease caused or mediated by B cells infected with Ebola virus (EBV)). In some embodiments, the immune disease is selected from the group consisting of rheumatoid arthritis, systemic lupus erythematosus, asthma, inflammatory bowel disease, multiple sclerosis, Crohn's disease, gastritis, Hashimoto's thyroiditis, ankylosing spondylitis and graft versus host disease. In some embodiments, the immune disease is rheumatoid arthritis.

The anti-CD38 antibodies or the antigen-binding fragments thereof of the present disclosure exhibit favorable efficiency in both biochemical tests and in vivo pharmacodynamic assays. In the test for detecting the affinity of antibody-to-antigen, the antibody of the present disclosure hu9E showed KD value of 1.31 nM to human CD38, hu11E showed KD value of 0.568 nM to human CD38, and hu160E showed KD value of 0.0585 nM to human CD38, whereas the control antibody showed KD value of 2.35 nM, suggesting that the antibodies of the present disclosure have higher affinity (Table 18).

In the test of in vivo inhibition of tumor, it was found that both antibodies hu11E and hu160E of the present disclosure can effectively inhibit tumor growth in mice. The tumor-inhibition rate of hu11E at a dosage of 1 mpk was up to 93.14%, and hu160E exhibited tumor-inhibition rate of 70.02%, both were significantly higher than that of control antibody Dara (the tumor-inhibition rate was 56.83%) (Table 20).

In addition, the anti-CD38 monoclonal antibodies or the antigen-binding fragments thereof of the present disclosure have favorable metabolic kinetic properties in rats, and show a longer half-life and higher bioavailability.

### DESCRIPTION OF THE DRAWINGS

FIG. 1A: In vitro ADCP test results of CD38 antibodies on Molp-8 cells.

FIG. 1B: In vitro ADCP test results of CD38 antibodies on Daudi cells.

FIG. 2: Tumor-inhibition effects of CD38 antibodies in mice.

### DETAILED DESCRIPTION OF THE INVENTION

### Terminology

In order to make the present disclosure be more easily understood, certain technical and scientific terms are specifically defined below. Unless otherwise defined explicitly herein, all other technical and scientific terms used herein have the meaning commonly understood by those skilled in the art to which this disclosure pertains.

Three-letter codes and one-letter codes for amino acids used in the present disclosure are as described in J. biol. chem, 243, p3558 (1968).

As used herein, “antibody” refers to immunoglobulin, a four-peptide chain structure connected together by disulfide bond between two identical heavy chains and two identical light chains. Different immunoglobulin heavy chain constant regions exhibit different amino acid compositions and sequences, hence present different antigenicity. Accordingly, immunoglobulins can be divided into five types (or immunoglobulin isotypes), namely IgM, IgD, IgG, IgA and IgE, corresponding to heavy chain μ, δ, γ, α and ε, respectively According to its amino acid composition of hinge region and the number and location of heavy chain disulfide bonds, the same type of Ig can further be divided into different sub-types, for example, IgG can be divided into IgG1, IgG2, IgG3 and IgG4. Light chain can be divided into κ or λ chain based on different constant regions. Each of five types of Ig has κ or λ chain.

About 110 amino acid sequences adjacent to the N-terminus of the antibody heavy and light chains are highly variable, known as variable region (Fv region); the rest of amino acid sequences close to the C-terminus are relatively stable, known as constant region. The variable region includes three hypervariable regions (HVRs) and four relatively conserved framework regions (FRs). The three hypervariable regions which determine the specificity of the antibody are also known as complementarity determining regions (CDRs). Each light chain variable region (VL or LCVR) and each heavy chain variable region (VH or HCVR) consists of three CDR regions and four FR regions, with sequential order from the amino terminus to carboxyl terminus as the following: FR1, CDR1, FR2, CDR2, FR3, CDR3, and FR4. The three CDR regions of the light chain refer to LCDR1, LCDR2, and LCDR3, and the three CDR regions of the heavy chain refer to HCDR1, HCDR2, and HCDR3.

The antibodies of the present disclosure include murine antibodies, chimeric antibodies, and humanized antibodies, preferably humanized antibodies.

As used herein, the term “murine antibody” refers to anti-human CD38 monoclonal antibodies prepared according to the knowledge and skills in the art. During the preparation, test subject is injected with CD38 antigen, and then a hybridoma expressing the antibody which possesses desired sequence or functional characteristics is isolated. In a preferable embodiment of the present disclosure, the murine CD38 antibody or antigen-binding fragment thereof further comprises a light chain constant region of murine κ, λ chain or variant thereof, or further comprises a heavy chain constant region of murine IgG1, IgG2, IgG3 or variant thereof.

The term “chimeric antibody” is an antibody obtained by fusing the variable region of a species (such as murine) antibody with the constant region of another species (such as human) antibody, and the chimeric antibody can alleviate the murine antibody-induced immune response. To prepare a chimeric antibody, a hybridoma secreting specific murine monoclonal antibody is firstly prepared and variable region gene is cloned from the murine hybridoma; then constant region gene is cloned from human antibody according to the need; the murine variable region gene is connected to the human constant region gene to form a chimeric gene, which can be subsequently inserted into an expression vector. Finally the chimeric antibody molecule will be expressed in eukaryotic or prokaryotic system. In a specific embodiment of the present disclosure, the antibody light chain of the CD38 chimeric antibody further comprises a light chain constant region of a human kappa, lambda chain or a variant thereof. The antibody heavy chain of the CD38 chimeric antibody further comprises a heavy chain constant region of human IgG1, IgG2, IgG3, IgG4 or a conventional variant thereof, preferably comprises a heavy chain constant region of human IgG1, and more preferably comprises an IgG1 heavy chain constant region with amino acid mutations (such as E333A mutation) which enhance the CDC function.

The term “humanized antibody” refers to an antibody generated by grafting murine CDR sequences into human antibody variable region frameworks, i.e., an antibody produced in different types of human germline antibody framework sequences. Humanized antibody can avoid heterologous responses induced by chimeric antibody which carries a large number of murine protein components. Such framework sequences can be obtained from public DNA database or published references covering sequences of germline antibody gene. For example, germline DNA sequences of human heavy and light chain variable region genes can be found in “VBase” human germline sequence database (www.mrccpe.com.ac.uk/vbase), as well as in Kabat, E A, et al. 1991 Sequences of Proteins of Immunological Interest, 5th Ed. To avoid a decrease in activity caused by the decreased immunogenicity, the framework sequences in human antibody variable region may be subjected to minimal reverse mutations or back mutations to maintain the activity. The humanized antibodies of the present disclosure also include humanized antibodies obtained after CDR affinity maturation by phage display or yeast display.

The grafting of CDR can result in the decrease of the affinity of the antibody or antigen-binding fragment thereof to the antigen, due to the change of the framework residues responsible for the contact with the antigen. Such interactions may be resulted from highly somatic mutations. Therefore, it is necessary to graft the donor framework amino acids to the humanized antibody framework. The amino acid residues involved in antigen binding derived from non-human antibody or antigen-binding fragment thereof can be identified by checking the sequence and structure of animal monoclonal antibody variable region. The different amino acid residues between the donor CDR framework and the germlines may be considered to be related. If it is not possible to determine the most closely related germline, the sequence may be aligned against the consensus sequence shared among subtypes or against the murine sequence having high similarity percentage. Rare residues in framework are thought to be the result of a mutation in somatic cells, and play an important role in binding.

In some specific embodiments of the present disclosure, the antibody light chain of the CD38 humanized antibody further comprises a light chain constant region of a human kappa, lambda chain or a conventional variant thereof. The antibody heavy chain of the CD38 humanized antibody further comprises a heavy chain constant region of human IgG1, IgG2, IgG3, IgG4 or a conventional variant thereof, preferably comprises a heavy chain constant region of human IgG1, and more preferably comprises an IgG1 heavy chain constant region with amino acid mutations (such as E333A mutation) which enhance the CDC function.

The “conventional variants” of the human antibody heavy chain constant region and the antibody light chain constant region described in the present disclosure refer to the human heavy or light chain constant region variants disclosed in the prior art which do not change the structure and function of the antibody variable regions. Exemplary variants include IgG1, IgG2, IgG3 or IgG4 heavy chain constant region variants by site-directed modification and amino acid substitutions on the heavy chain constant region. The specific substitutions are, for example, YTE mutation, L234A and/or L235A mutation, S228P mutation, E333A mutation, and/or mutations resulting in a knob-into-hole structure (making the antibody heavy chain have a combination of knob-Fc and hole-Fc), etc. These mutations have been proven to confer the antibody with new properties, without affecting the function of the antibody variable region.

The term “back-mutation” refers to reversion of the amino acid residues in FR regions derived from human antibody back to the amino acid residues corresponding to those from the original antibody. In order to avoid the decrease in activity caused by the humanized antibody, usually the variable regions of the humanized antibodies can be subjected to minimal reverse mutations to maintain the activity of the antibody.

“Human antibody (HuMAb)”, “antibody derived from human”, “full human antibody” and “complete human antibody” may be used interchangeably, and may be antibodies derived from human or antibodies obtained from a genetically modified organism which has been “engineered” by any method known in the art to produce specific human antibodies in response to antigen stimulation. In some technologies, elements of human heavy and light chain loci are introduced into organism cell strains derived from embryonic stem cell lines, in which the endogenous heavy and light chain loci have been knocked out specifically. Transgenic organisms can synthesize human antibodies specific for human antigens, and the organisms can be used to produce hybridomas that secrete human antibodies. A human antibody can also be such antibody in which the heavy and light chains are encoded by nucleotide sequences derived from one or more DNA of human sources. Full human antibodies can also be constructed by gene or chromosome transfection methods and phage display technology, or constructed from B cells activated in vitro, all of methods are known in the art.

The terms “full-length antibody”, “full antibody”, “whole antibody” and “complete antibody” are used interchangeably herein and refer to an antibody in a substantially complete form, which is distinguished from the antigen-binding fragment defined below. The term specifically refers to antibodies that contain constant regions in the light and heavy chains.

In some embodiments, the full-length antibodies of the present disclosure include full-length antibodies formed by linking the light chain variable region to the light chain constant region, and the heavy chain variable region to the heavy chain constant region, as shown in Table 1 below. Those skilled in the art can select the light chain constant region and heavy chain constant region from various antibody sources according to need, such as human antibody-derived light chain constant region (or conventional variant thereof) and heavy chain constant region (or conventional variant thereof). At the same time, the combination of the light and heavy chain variable regions described in Table 1 can form a single chain antibody (scFv), Fab, or other forms of antigen-binding fragments comprising scFv or Fab.

TABLE 1
Combinations of light chain variable region and heavy
chain variable region of anti-CD38 humanized antibody

| Combination | Heavy chain | Light chain |
|---|---|---|
| of variable | variable | variable |
| regions | region VH | region VL |
| h009-01V | h009 VH1 | h009 VL1 |
| h009-02V | h009 VH2 | h009 VL1 |
| h009-03V | h009 VH3 | h009 VL1 |
| h009-04V | h009 VH4 | h009 VL1 |
| h009-05V | h009 VH5 | h009 VL1 |
| h009-06V | h009 VH1 | h009 VL2 |
| h009-07V | h009 VH2 | h009 VL2 |
| h009-08V | h009 VH3 | h009 VL2 |
| h009-09V | h009 VH4 | h009 VL2 |
| h009-10V | h009 VH5 | h009 VL2 |
| h009-11V | h009 VH1 | h009 VL3 |
| h009-12V | h009 VH2 | h009 VL3 |
| h009-13V | h009 VH3 | h009 VL3 |
| h009-14V | h009 VH4 | h009 VL3 |
| h009-15V | h009 VH5 | h009 VL3 |
| h011-01V | h011 VH1 | h011VL1 |
| h011-02V | h011 VH2 | h011VL2 |
| h011-03V | h011 VH1 | h011VL3 |
| h011-04V | h011 VH2 | h011VL1 |
| h011-05V | h011 VH1 | h011VL2 |
| h011-06V | h011 VH2 | h011VL3 |
| h160-01V | h160 VH1 | h160 VL1 |
| h160-02V | h160 VH1 | h160 VL2 |
| h160-03V | h160 VH1 | h160 VL3 |
| h160-04V | h160 VH1 | h160 VL4 |
| h160-05V | h160 VH2 | h160 VL1 |
| h160-06V | h160 VH2 | h160 VL2 |
| h160-07V | h160 VH2 | h160 VL3 |
| h160-08V | h160 VH2 | h160 VL4 |

Note:
For example, ″h009-01V” represents the light/heavy chain variable region pair of h009-01V, in which the heavy chain variable region is h009 VH1 (SEQ ID No: 24), and the light chain variable region is h009 VL1 (SEQ ID No: 25), and so on.

The term “monoclonal antibody” refers to an antibody obtained from a population of substantially homogeneous antibodies, that is, the individual antibodies constituting the population are identical and/or bind to the same epitope, except for possible variant antibodies (for example, variants containing naturally occurring mutations or mutations produced during the manufacture of monoclonal antibody preparations, which are usually present in minimal amounts). Unlike polyclonal antibody preparations that usually contain different antibodies directed against different determinants (epitopes), each monoclonal antibody of a monoclonal antibody preparation (formulation) is directed against a single determinant on the antigen. Therefore, the prefix “monoclonal” indicates the characteristics of the antibody obtained from a substantially homogeneous antibody population, and should not be interpreted as antibody manufactured by particular method. For example, monoclonal antibodies used in accordance with the present disclosure can be prepared by various techniques, including but not limited to hybridoma methods, recombinant DNA methods, phage display methods, and methods by using transgenic animals containing all or part of human immunoglobulin loci. Such methods, and other exemplary methods for preparing monoclonal antibodies are described herein.

The term “antigen-binding fragment” or “functional fragment” of an antibody refers to one or more fragments of the antibody that retain the ability to specifically bind to an antigen (e.g., CD38). It has been shown that fragments of a full-length antibody can be used to achieve function of binding to a specific antigen. Examples of the binding fragments contained in the term “antigen-binding fragment” of an antibody include (i) Fab fragment, a monovalent fragment composed of VL, VH, CL and CH1 domains; (ii) F(ab′)₂ fragment, a bivalent fragment formed by two Fab fragments connected by a disulfide bridge at the hinge region, (iii) Fv fragment composed of the VH and VL domains of one arm of the antibody; (iv) dsFv, a stable antigen-binding fragment formed by VH and VL via interchain disulfide bond(s); and (v) diabody, bispecific antibody and multispecific antibody containing fragments such as scFv, dsFv, and Fab. In addition, the VL domain and VH domain in a Fv fragment are encoded by two separate genes; however they can be linked by a synthetic linker by using recombinant methods, to generate a single protein chain in which a monovalent molecular is formed by pairing the VL and VH domain (referred to as single chain Fv (scFv); see, e.g., Bird et al. (1988) Science 242: 423-426; and Huston et al (1988) Proc. Natl. Acad. Sci USA 85:5879-5883). Such single chain antibodies are also intended to be included in the term “antigen-binding fragment”. Such antibodie fragments are obtained using conventional techniques known in the field, and screened for functional fragments by using the same method as that for an intact antibody. Antigen binding portions can be produced by recombinant DNA technology or by enzymatic or chemical digestion of an intact immunoglobulin. Antibodies can be in the form of different isotypes, e.g., IgG (e.g., IgG1, IgG2, IgG3 or IgG4 subtype), IgA1, IgA2, IgD, IgE or IgM antibody. In some embodiments, the antigen-binding fragments thereof of the present disclosure include Fab, F(ab′)2, Fab′, single-chain antibody (scFv), dimerized V region (diabody), V region stabilized by disulfide linkage (dsFv), and so on.

Fab is an antibody fragment obtained by treating an IgG antibody molecule with a papain (which cleaves the amino acid residue at position 224 of the H chain). The obtained fragment has a molecular weight of about 50,000 and has antigen binding activity, in which about a half of H chain at the N-terminal side and the entire L chain are bound together through disulfide bond(s).

Fab of the present disclosure can be produced by treating the monoclonal antibody of the present disclosure with papain. Further, the Fab can be produced by inserting DNA encoding Fab of the antibody into a prokaryotic expression vector or eukaryotic expression vector, and introducing the vector into a prokaryote or eukaryote to express the Fab.

“F(ab′)2” refers to an antibody fragment with a molecular weight of about 100,000 and antigen-binding activity, which is obtained by digesting IgG by pepsin at the part downstreaming the two disulfide bonds in the hinge region. F(ab′)2 contains two Fabs connected at the hinge region.

F(ab′)2 of the present disclosure can be produced by treating the monoclonal antibody of the present disclosure with pepsin. Also, F(ab′)2 can be produced by binding the Fab′ described below via thioether bond or disulfide bond.

“Fab′” is an antibody fragment having a molecular weight of about 50,000 and having antigen binding activity, which is obtained by cleaving a disulfide bond at the hinge region of the F(ab′)2 described above. The Fab′ of the present disclosure can be produced by treating F(ab′)2 of the present disclosure with a reducing agent such as dithiothreitol. Further, the Fab′ can be produced by inserting DNA encoding Fab′ of the antibody into a prokaryotic expression vector or eukaryotic expression vector and introducing the vector into a prokaryote or eukaryote to express the Fab′.

The term “single chain antibody”, “single chain Fv” or “scFv” refers to a molecule comprising antibody heavy chain variable domain (or region; VH) connected to antibody light chain variable domain (or region; VL) by a linker. Such scFv molecules have general structure of NH₂-VL-linker-VH-COOH or NH₂-VH-linker-VL-COOH. A suitable linker in the prior art consists of repeated GGGGS amino acid sequence or variant thereof, for example, variant with 1-4 repeats (Holliger et al. (1993), Proc. Natl. Acad. Sci. USA 90:6444-6448). Other linkers that can be used in the present disclosure are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol.

The scFv of the present disclosure can be produced by the following steps: obtaining cDNAs encoding the VH and VL of the monoclonal antibody of the present disclosure, constructing a DNA encoding the scFv, inserting the DNA into a prokaryotic or eukaryotic expression vector, and then introducing the expression vector into a prokaryote or eukaryote to express the scFv.

“Diabody” is an antibody fragment wherein the scFv is dimerized, and it is an antibody fragment having divalent antigen binding activity. In the divalent antigen binding activity, the two antigens may be the same or different.

Bispecific and multispecific antibody refer to an antibody that can simultaneously bind to two or more antigens or antigenic determinants, including scFv or Fab fragments that can bind to CD38.

The diabody of the present disclosure can be produced by the following steps: obtaining cDNAs encoding VH and VL of the monoclonal antibody of the present disclosure, constructing a DNA encoding scFv to make the length of a linker peptide of 8 or less amino acid residues, inserting the DNA into a prokaryotic or eukaryotic expression vector, and then introducing the expression vector into a prokaryote or eukaryote to express the diabody.

“dsFv” is obtained by substituting one amino acid residue in each of VH and VL with a cysteine residue, and then connecting the substituted polypeptides via a disulfide bond between the two cysteine residues. The amino acid residues to be substituted with a cysteine residue can be selected based on three-dimensional structure prediction of the antibody in accordance with known methods (Protein Engineering, 7, 697 (1994)).

The dsFv of the present disclosure can be produced by the following steps: obtaining cDNAs encoding the VH and VL of the monoclonal antibody of the present disclosure, constructing a DNA encoding the dsFv, inserting the DNA into a prokaryotic or eukaryotic expression vector, and then introducing the expression vector into a prokaryote or eukaryote to express the dsFv.

As used herein, the term “framework (FR)” refers to a part of the variable domain (either VL or VH), which serves as a scaffold for the antigen-binding loops (CDRs) in the variable domain. Essentially, it is a variable domain without CDRs.

The term “amino acid difference” or “amino acid mutation” refers to the difference or mutation between a polypeptide and its variant, and refers to the amino acid change or mutation present in the protein or polypeptide variant when compared to the original protein or polypeptide, including 1, 2, 3 or more amino acid substitution(s), insertion(s) or deletion(s) on the basis of the original protein or polypeptide.

The term “complementarity determining region”, “CDR” or “hypervariable region” refers to one of the six hypervariable regions present in the antibody variable domain that mainly contribute to antigen binding. Generally, there are three CDRs (HCDR1, HCDR2, HCDR3) in each heavy chain variable region, and three CDRs (LCDR1, LCDR2, LCDR3) in each light chain variable region. The amino acid sequence boundaries of CDRs can be determined by any well-known criteria, including the “Kabat” numbering criteria (see Kabat et al. (1991), “Sequences of Proteins of Immunological Interest”, 5th edition, Public Health Service, National Institutes of Health, Bethesda, Md.), “Chothia” numbering criteria (see Al-Lazikani et al., (1997) JMB 273:927-948) and ImMunoGenTics (IMGT) numbering criteria (Lefranc M P, Immunologist, 7, 132-136 (1999); Lefranc, M P, etc., Dev. Comp. Immunol., 27, 55-77 (2003), and the like. For example, for the classical format, the Kabat criteria can be followed, the CDR amino acid residues in the heavy chain variable domain (VH) are numbered as 31-35 (HCDR1), 50-65 (HCDR2) and 95-102 (HCDR3); the CDR amino acid residues in the light chain variable domain (VL) are numbered as 24-34 (LCDR1), 50-56 (LCDR2), and 89-97 (LCDR3). Following the Chothia criteria, the CDR amino acid residues in VH are numbered as 26-32 (HCDR1), 52-56 (HCDR2) and 95-102 (HCDR3); and the amino acid residues in VL are numbered as 26-32 (LCDR1), 50-52 (LCDR2) and 91-96 (LCDR3). By combining both Kabat and Chothia to define CDR, CDRs are composed of amino acid residues 26-35 (HCDR1), 50-65 (HCDR2) and 95-102 (HCDR3) in the human VH and amino acid residues 24-34 (LCDR1), 50-56 (LCDR2) and 89-97 (LCDR3) in the human VL. Following IMGT criteria, the CDR amino acid residues in VH are roughly numbered as 26-35 (CDR1), 51-57 (CDR2) and 93-102 (CDR3), and the CDR amino acid residues in VL are roughly numbered as 27-32 (CDR1), 50-52 (CDR2) and 89-97 (CDR3). Following IMGT criteria, the CDR regions of an antibody can be determined using IMGT/DomainGap Align Program.

The term “epitope” or “antigenic determinant” refers to a site on an antigen to which an immunoglobulin or antibody specifically binds (e.g., a specific site on CD38 molecule). Epitopes typically include at least 3, 4, 5, 6, 7, 8, 9, 10, 11, 12, 13, 14 or 15 contiguous or non-contiguous amino acids in a unique spatial conformation. See, for example, Epitope Mapping Protocols in Methods in Molecular Biology, Vol. 66, G. E. Morris Ed. (1996).

The term “specifically bind to” or “selectively bind to” refers to the binding of an antibody to a predetermined epitope on an antigen. Typically, the antibody binds with an affinity (KD) of less than about 10⁻⁸M, for example, less than about 10⁻⁹ M, 10⁻¹⁰ M or 10⁻¹¹ M or even less.

The term “KD” refers to the dissociation equilibrium constant for particular antibody-antigen interaction. Typically, the antibody of the present disclosure binds to CD38 with a dissociation equilibrium constant (KD) of less than about 10⁻⁷M, for example, less than about 10⁻⁸M, 10⁻⁹M or 10⁻¹⁰M or even less, for example, as determined by Surface Plasma Resonance (SPR) technology in BIACORE instrument.

When the term “competition” is used in the context of antigen-binding proteins (e.g., neutralizing antigen-binding proteins or neutralizing antibodies) that compete for the same epitope, it means that competition occurs between the antigen-binding proteins, which is determined by an assay wherein an antigen-binding protein to be tested (e.g., an antibody or antigen-binding fragment thereof) prevents or inhibits (e.g., reduces) the specific binding of a reference antigen-binding protein (e.g., a ligand or reference antibody) to a common antigen (e.g., a CD38 antigen or fragment thereof). Numerous types of competitive binding assays are available to determine whether an antigen-binding protein competes with another.

These assays are, for example, solid phase direct or indirect radioimmunoassay (RIA), solid phase direct or indirect enzyme immunoassay (EIA), Sandwich competition assay (see, e.g., Stahli et al, 1983, Methods in Enzymology 9: 242-253); solid phase direct biotin-avidin EIA (see, e.g., Kirkland et al, 1986, J. Immunol. 137: 3614-3619; Cheung et al., 1990, Virologyl76: 546-552), solid phase direct labeling assay, solid phase direct labeling sandwich assay (see, e.g., Harlow and Lane, 1988, Antibodies, A Laboratory Manual, Cold Spring Harbor Press); solid phase direct labeling MA with I-125 label (see, e.g., Morel et al, 1988, Molec. Immunol. 25: 7-15); and direct labeling MA (Moldenhauer et al, 1990, Scand. J. Immunol. 32: 77-82). Typically, the assay involves the use of a purified antigen capable of binding to both an unlabeled test antigen-binding protein and a labeled reference antigen-binding protein (the antigen is on a solid surface or cell surface). Competitive inhibition is determined by measuring the amount of label bound to the solid surface or to the cell surface in the presence of the test antigen-binding protein. Usually, the test antigen-binding protein is present in excess. Antigen binding proteins identified by competitive assay (competitive antigen-binding protein) includes: antigen-binding proteins that bind to the same epitope as the reference antigen-binding protein; and antigen-binding proteins that bind to an epitope that is sufficiently close to the epitope to which the reference antigen-binding protein binds, where the two epitopes spatially interfere with each other, thereby interfering the binding. Additional details regarding methods for determining competitive binding are provided in the Examples herein. Typically, when a competitive antigen-binding protein is present in excess, it will inhibit (e.g., reduce) at least 40-45%, 45-50%, 50-55%, 55-60%, 60-65%, 65-70%, 70-75% or even more of the specific binding of the reference antigen-binding protein to the common antigen. In some cases, the binding is inhibited by at least 80-85%, 85-90%, 90-95%, 95-97% or even more.

As used herein, the term “nucleic acid molecule” refers to DNA molecules and RNA molecules. A nucleic acid molecule may be single-stranded or double-stranded, but preferably is double-stranded DNA. A nucleic acid is “operably linked” when it is placed into a functional relationship with another nucleic acid sequence. For instance, a promoter or enhancer is operably linked to a coding sequence, when it affects the transcription of the sequence.

The term “vector” or “expression vector” refers to a nucleic acid molecule capable of transporting another nucleic acid to which it has been linked. In one embodiment, the vector is a “plasmid,” which refers to a circular double stranded DNA loop into which additional DNA segments may be ligated. In another embodiment, the vector is a viral vector, wherein additional DNA segments may be ligated into the viral genome. The vectors disclosed herein are capable of self-replicating in the host cell into which they are introduced (e.g., bacterial vectors having a bacterial replication origin and episomal mammalian vectors), or may be integrated into the genome of a host cell upon introduction into the host cell, and thereby are replicated along with the host genome (e.g., non-episomal mammalian vectors).

Methods for producing and purifying antibodies and antigen-binding fragments thereof are well known in the art, for example, A Laboratory Manual for Antibodies, Cold Spring Harbor, N.Y., chapters 5-8 and 15. For example, mice can be immunized with human CD38 or fragments thereof, and the resulting antibodies can then be renatured, purified, and sequenced for amino acid sequences by using conventional methods well known in the art. Antigen-binding fragments can also be prepared by conventional methods. The antibodies or the antigen-binding fragments thereof of the present disclosure are engineered to graft one or more human FR regions onto CDRs derived from non-human antibody. Human FR germline sequences can be obtained from ImMunoGeneTics (IMGT) via their website http://imgt.cines.fr, or from The Immunoglobulin Facts Book, 2001, ISBN 012441351, by aligning against IMGT human antibody variable germline gene database using MOE software.

The term “host cell” refers to a cell into which an expression vector has been introduced. Host cells may include microorganisms (such as bacteria), plants or animal cells. Bacteria susceptible to be transformed include members of the family Enterobacteriaceae, such as strains of Escherichia coli or Salmonella; the family Bacillaceae such as Bacillus subtilis; Pneumococcus; Streptococcus and Haemophilus influenzae. Suitable microorganisms include Saccharomyces cerevisiae and Pichia pastoris. Suitable animal host cell lines include CHO (Chinese Hamster Ovary Cell Line) and NS0 cells.

The engineered antibodies or the antigen-binding fragments thereof of the present disclosure may be prepared and purified using known methods. For example, cDNA sequences encoding a heavy chain and a light chain may be cloned and engineered into a GS expression vector. The vectors expressing recombinant immunoglobulin may then be stably transfected into CHO cells. As a more recommended method well known in the art, mammalian expression systems will result in glycosylation, typically at highly conserved N-terminal sites in the Fc region. Stable clones expressing an antibody specifically binding to human CD38 were obtained. Positive clones may be expanded in serum-free culture medium in bioreactors for antibody production. Culture medium, into which an antibody has been secreted, may be purified by conventional techniques. For example, purification may be performed on Protein A or G Sepharose FF column that has been modified with buffer. The nonspecific binding components are removed by washing. The bound antibody is eluted by pH gradient and antibody fragments are detected by SDS-PAGE, and then pooled. The antibodies may be filtered and concentrated using common techniques. Soluble mixtures and aggregates may be effectively removed by common techniques, such as size exclusion or ion exchange. The resulting product is sometimes needed to be frozen immediately, such as at −70° C., or lyophilized.

“Administration”, “administering” or “treatment,” as it applies to an animal, human, subject, cell, tissue, organ, or biological fluid, refers to contacting an exogenous pharmaceutical, therapeutic, diagnostic agent, or composition with the animal, human, subject, cell, tissue, organ, or biological fluid. “Administration”, “administering” or “treatment” can refer, e.g., to therapeutic, pharmacokinetic, diagnostic, research, and experimental methods. Treatment of a cell involves contacting a reagent with the cell, as well as contacting a reagent with a fluid, where the fluid is in contact with the cell. “Administration”, “administering” or “treatment” also means in vitro or ex vivo treatments, e.g., of a cell, with a reagent, diagnostic, binding compound, or with another cell. “Treatment”, as it applies to a human, veterinary, or research subject, refers to therapeutic treatment, prophylactic or preventative measures, to research and diagnostic applications.

“Treatment” means administering a therapeutic agent, such as a composition containing any of antibodies or the antigen-binding fragments thereof of the present disclosure, or a nucleic acid molecule encoding the antibody or the antigen-binding fragment thereof, internally or externally to a patient having one or more disease symptoms for which the therapeutic agent is known to have therapeutic effect. Typically, the agent is administered in an amount effectively to alleviate one or more disease symptoms in the patient or population to be treated, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to various factors such as the disease state, age, and body weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While one embodiment of the present disclosure (e.g., a treatment method or article of manufacture) may not be effective in alleviating each target disease symptom, it should alleviate the target disease symptom(s) in a statistically significant number of subjects as determined by any statistical test known in the art such as Student's t-test, chi-square test, U-test according to Mann and Whitney, Kruskal-Wallis test (H-test), Jonckheere-Terpstra-test and Wilcoxon-test.

“Conservative modification” or “conservative substitution or replacement” refers to substitution of amino acid(s) in a protein with other amino acid(s) having similar characteristics (e.g. charge, side-chain size, hydrophobicity/hydrophilicity, backbone conformation and rigidity, etc.), such that the changes can frequently be made without affecting the biological activity of the protein. Those skilled in the art recognize that, in general, single amino acid substitution in non-essential regions of a polypeptide does not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 “Exemplary Amino Acid Conservative Substitutions” below.

TABLE 2
Exemplary amino acid conservative substitutions

| Original residue | Conservative substitution |
|---|---|
| Ala (A) | Gly; Ser |
| Arg (R) | Lys; His |
| Asn (N) | Gln; His; Asp |
| Asp (D) | Glu; Asn |
| Cys (C) | Ser; Ala; Val |
| Gln (Q) | Asn; Glu |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Effective amount” or “effective dosage” refers to the amount of the agent, compound, or pharmaceutical composition necessary to obtain any one or more beneficial or desired results. For prophylactic applications, beneficial or desired results include elimination or reduction of risk, reduction of severity, or delay of the onset of the disease, including the biochemical, histological, and behavioral symptoms of the condition, its complications, and intermediate pathological phenotypes during the development of the condition. For therapeutic applications, beneficial or desired results include clinical results, such as reduction of the incidence of various conditions associated with target antigen of the present disclosure or improvement of one or more symptoms of the condition, reduction of the dosage of other agents required to treat the condition, enhancement of the efficacy of another agent, and/or delay of the progression of the condition associated with the target antigen of the present disclosure in patients.

“Exogenous” refers to substances produced outside organisms, cells, or humans according to circumstances.

“Endogenous” refers to substances produced inside organisms, cells, or human bodies according to circumstances.

The “mutated sequence” mentioned in the present disclosure refers to the nucleotide sequence and amino acid sequence of varying percentage sequence identity to those of the present disclosure, which are obtained after modifying the nucleotide sequence and amino acid sequence of the present disclosure by appropriate substitution, insertion or deletion. The sequence identity described in the present disclosure may be at least 85%, 90% or 95%, non-limiting examples include 85%, 86%, 87%, 88%, 89%, 90%, 91%, 92%, 93%, 94%, 95%, 96%, 97%, 98%, 99%, 100%.

As used herein, “homology” or “identity” refers to sequence similarity between two polynucleotide sequences or between two polypeptide sequences. When a position in both of the two sequences to be compared is occupied by the same base or amino acid monomer subunit, e.g., when a position in each of two DNA molecules is occupied by the same base, then the molecules are known as homologous at that position. The percent of homology between two sequences is a function of the number of matching or homologous positions shared by the two sequences divided by the number of total positions to be compared and then multiplied by 100. For example, when two sequences are optimally aligned, if 6 out of 10 positions in the two sequences are matched or homologous, then the two sequences are 60% homologous; if 95 out of 100 positions in the two sequences re matched or homologous, then the two sequences are 95% homologous. Generally, the two sequences to be compared are subjected to alignment to give a maximum homology percentage. For example, the comparison can be performed by the BLAST algorithm, in which the parameters of the algorithm are selected to give the maximum match between each sequence over the entire length of each reference sequence. The following references refer to the BLAST algorithm frequently used for sequence analysis: BLAST algorithm (BLAST ALGORITHMS): Altschul, S F et al., (1990) J. Mol. Biol. 215:403-410; Gish, W. et al., (1993) Nature Genet. 3:266-272; Madden, T L et al., (1996) Meth. Enzymol. 266:131-141; Altschul, S F et al., (1997) Nucleic Acids Res. 25:3389-3402; Zhang, J. et al. (1997) Genome Res. 7:649-656. Other conventional BLAST algorithms such as those available from NCBI BLAST are also well known to those skilled in the art.

As used herein, the expressions “cell,” “cell line,” and “cell culture” are used interchangeably and all such designations include progeny. Thus, “transformant” and “transformed cell” include the primary subject cells and cultures derived therefrom regardless of the number of passages. It should be also understood that all progeny may not be precisely identical in DNA content, due to intended or non-intended mutations. Mutant progeny that have the same function or biological activity as screened in the originally transformed cells are included. Where different designations are intended to, it will be clearly understood from the context.

As used herein, “polymerase chain reaction” or “PCR” refers to a procedure or technique in which minute amounts of a specific portion of nucleic acid, RNA and/or DNA, are amplified as described in, e.g., U.S. Pat. No. 4,683,195. Generally, sequence information at the ends of or beyond the region of interest needs to be available, such that oligonucleotide primers can be designed; these primers will be identical to or similar to the sequence of complementary strand of the template to be amplified. The 5′ terminal nucleotides of the two primers can be identical to the ends of the amplified material. PCR can be used to amplify specific RNA sequences, specific DNA sequences from total genome and cDNA transcribed from total cellular RNA, bacteriophage or plasmid sequences, etc. See generally Mullis et al. (1987) Cold Spring Harbor Symp. Ouant. Biol. 51:263; Erlich editor, (1989) PCR TECHNOLOGY (Stockton Press, NY). The PCR used in the present disclosure is considered to be one (but not the only) example of polymerase reaction methods for amplifying a nucleic acid sample to be tested. The method comprises the use of nucleic acid sequences known as primers together with nucleic acid polymerase to amplify or generate a specific portion of nucleic acid.

“Isolated” refers to a purified state, in which the designated molecule is substantially free of other biological molecules, such as nucleic acids, proteins, lipids, carbohydrates, or other materials, such as cell debris and growth medium. In general, the term “isolated” is not intended to mean the complete absence of these materials or the absence of water, buffers or salts, unless they are present in an amount that significantly interferes with the experimental or therapeutic use of the compound as described herein. “Optional” or “optionally” means that the event or circumstance that follows may but does not necessarily occur, and the description will indicate the instances where the event or circumstance does or does not occur. For example, “optionally contains 1-3 antibody heavy chain variable regions” means the antibody heavy chain variable region with specific sequence can be, but need not be, present. “Pharmaceutical composition” refers to a mixture containing one or more antibodies or the antigen-binding fragments thereof according to the present disclosure and other chemical components, such as physiologically/pharmaceutically acceptable carriers or excipients. The pharmaceutical composition aims at promoting the administration to an organism, facilitating the absorption of the active ingredient and thereby exerting a biological effect.

The term “pharmaceutically acceptable carrier” refers to any inactive substance suitable for use in a formulation for the delivery of antibodies or antigen-binding fragments. The carrier can be an anti-adhesive agent, binder, coating agent, disintegrating agent, filler or diluent, preservative (such as antioxidant, antibacterial or antifungal agent), sweetener, absorption delaying agent, wetting agent, emulsifier, buffer, and the like. Examples of suitable pharmaceutically acceptable carriers include water, ethanol, polyols (such as glycerol, propylene glycol, polyethylene glycol, and the like) dextrose, vegetable oil (such as olive oil), saline, buffer, buffered saline, and isotonic agent, such as sugars, polyols, sorbitol and sodium chloride.

“CD38-positive disease or disorder” is a disease or disorder in which CD38-expressing cells are present. Without limitation, regarding the immune diseases involving CD38-expressing B cells, plasma cells, monocytes and T cells, one characteristics of the disease is, for example, a tumor disease with CD38-expressing tumor cells, such as CD38-expressing leukemia, B cell lymphoma, plasma cell malignant tumor, T/NK cell lymphoma and myeloma. In some embodiments of the present disclosure, the leukemia is selected from the group consisting of acute lymphocytic leukemia, acute lymphoblastic leukemia, acute promyelocytic leukemia, chronic lymphocytic leukemia, acute and chronic myeloid leukemia. In some embodiments, the myeloma is selected from the group consisting of multiple myeloma, anterior medullary tumor, and light chain amyloidosis. In some embodiments, the lymphoma is non-Hodgkin's lymphoma or Hodgkin's lymphoma. In some embodiments, the tumor may be selected from B cell lymphoma/leukemia, including but not limited to: precursor B cell lymphoblastic leukemia/lymphoma, B cell non-Hodgkin's lymphoma or B cell Hodgkin Lymphoma, mature B cell tumor. In some embodiments, the tumor is selected from the group consisting of: B cell chronic lymphocytic leukemia (CLL), small lymphocytic leukemia (SLL), B cell acute lymphocytic leukemia, B cell prelymphocytic leukemia, lymphoplasmacytoid lymphoma, mantle cell lymphoma (MCL), follicular lymphoma (including low-grade, intermediate or high-grade FL), cutaneous follicular central lymphoma, marginal zone B cell lymphoma (including MALT type, lymph node MZBL type, spleen MZBL type), hairy cell leukemia, diffuse large B cell lymphoma, Burkitt lymphoma, plasma cell tumor, plasma cell myeloma, plasma cell leukemia, post-transplant lymphoproliferative disease, Waldenstrom macroglobulinemia, plasma cell leukemia, anaplastic large cell lymphoma (ALCL) and hairy cell lymphoma. In some embodiments, the tumor is multiple myeloma. In some embodiments, the immune disease may be selected from the group consisting of rheumatoid arthritis, psoriasis, ankylosing spondylitis, joint psoriasis, dermatitis, systemic scleroderma and sclerosis, inflammatory bowel disease (IBD), Crohn's disease, ulcerative colitis, respiratory distress syndrome, meningitis, encephalitis, gastritis, uveitis, glomerulonephritis, eczema, asthma, arteriosclerosis, leukocyte adhesion deficiency, Raynaud syndrome, Sjogren syndrome, juvenile diabetes, Reiter disease, Behcet disease, immune complex nephritis, IgA nephropathy, IgM polyneuropathy, immune-mediated thrombocytopenia symptom (e.g. acute idiopathic thrombocytopenic purpura, chronic idiopathic thrombocytopenic purpura), hemolytic anemia, myasthenia gravis, lupus nephritis, systemic lupus erythematosus, rheumatoid arthritis (RA), atopic dermatitis, pemphigus, Graves disease, Hashimoto's thyroiditis, Wegener's granulomatosis, Omenn syndrome, chronic renal failure, acute infectious mononucleosis, multiple sclerosis, HIV and herpes virus-related diseases, severe acute respiratory syndrome, chorioretinitis, graft versus host disease, and immune disease caused by virus infection (such as disease caused or mediated by B cells infected with Ebola virus (EBV)). In some embodiments, the immune disease is selected from the group consisting of rheumatoid arthritis, systemic lupus erythematosus, asthma, inflammatory bowel disease, multiple sclerosis, Crohn's disease, gastritis, Hashimoto's thyroiditis, ankylosing spondylitis and graft versus host disease. In some embodiments, the immune disease is rheumatoid arthritis.

In addition, the present disclosure provides agents for the treatment or prevention of diseases related to target antigen (e.g. CD38) positive cells. The agent contains the anti-CD38 antibody or the antigen-binding fragment thereof of the present disclosure as an active ingredient and a therapeutically or prophylactically effective amount of the agent can be administered to a subject in need for the treatment or prevention of CD38-positive diseases. The anti-CD38 antibodies or the antigen-binding fragments thereof can inhibit disease-related activities induced by CD38 or eliminate or reduce the number of CD38 expressing cells. The therapeutically or prophylactically effective amount of the composition comprises 0.1-3000 mg (preferably 0.1-2000 mg, more preferably 1-1000 mg) of the anti-CD38 antibody or the antigen-binding fragment thereof described above, in a unit dosage.

In addition, the present disclosure relates to methods for immunodetection or determination of target antigens (for example, CD38), reagents for immunodetection or determination of target antigens (for example, CD38), methods for immunodetection or determination of cells expressing target antigens (for example, CD38), and the diagnostic agents for diagnosing diseases associated with target antigen (for example, CD38)-positive cells, comprising the antibody or the antibody fragment of the present disclosure that specifically recognizes and binds to the target antigen (for example, human CD38), as an active ingredient.

In the present disclosure, the method for detecting or measuring the amount of the target antigen (e.g. CD38) may be any known method. For example, it includes immunoassay or immunodetection method.

The immunoassay or immunodetection method is a method of detecting or measuring the amount of an antibody or antigen with a labeled antigen or antibody. Examples of immunoassay or immunodetection methods include immunomethod using antibody labeled with radioactive substance (MA), enzyme immunoassay (EIA or ELISA), fluorescence immunoassay (FIA), luminescence immunoassay, western blotting, physicochemical method, and the like.

The diseases related to CD38-positive cells described above can be diagnosed by detecting or measuring CD38-expressing cells using the antibodies or the antibody fragments thereof of the present disclosure.

Cells expressing the polypeptide can be detected by the known immunodetection methods, preferably by immuno-precipitation, fluorescent cell staining, immunohistochemistry staining, and the like. In addition, the method, such as a staining method of fluorescent antibody using FMAT8100HTS system (Applied Biosystem), can be used.

In the present disclosure, samples to be detected or measured for the target antigen (e.g. CD38) are not particularly limited, as long as they are possible to contain cells expressing the target antigen (e.g. CD38), such as tissue cells, blood, plasma, serum, pancreatic secretion, urine, feces, tissue fluid or culture medium.

Dependent on the required diagnostic method, the diagnostic agent containing the monoclonal antibody or antibody fragment thereof of the present disclosure may also contain reagents for performing an antigen-antibody reaction or reagents for detecting the reaction. The reagents for performing an antigen-antibody reaction include buffers, salts and the like.

The reagents for detection include agents commonly used in immunoassay or immunodetection methods, for example, a labeled secondary antibody that recognizes the monoclonal antibody, antibody fragment or conjugate thereof, and a substrate corresponding to the label.

The details of one or more embodiments of the present disclosure are set forth in the description above. The preferred methods and materials are described below, although any method and material similar or identical to those described herein can be used in the practice or testing of the present disclosure. Through the specification and claims, other features, purposes and advantages of the present disclosure will become apparent.

In the specification and claims, the singular form also refers to its plural counterparts, unless the context clearly dictates otherwise. Unless otherwise defined explicitly herein, all technical and scientific terms used herein have the meaning commonly understood by those skilled in the art to which this disclosure pertains. All patents and publications cited in the specification are incorporated by reference. The following examples are provided to more fully illustrate the preferred embodiments of the present disclosure. These examples should not be construed as limiting the scope of the present disclosure in any way, and the scope of the present disclosure is defined by the claims.

### Examples and Test Examples

The following examples and test examples are provided to further describe the present disclosure, but are not intended to limit the scope of the disclosure. Experimental methods for which the specific conditions are not indicated in the examples and test examples of the present disclosure are generally carried out according to conventional conditions, such as Sambrook et al., Antibodies Laboratory Manual, Molecular Cloning, by Cold Spring Harbor Laboratory; or according to the conditions recommended by the manufacturer. Reagents for which the sources are not specifically indicated are commercially available reagents.

### Example 1. Preparation of CD38 Antigen

The amino acid sequences of the antigens and proteins for detection used in the present disclosure were designed using UniProt ADP-Ribocycliase/Cyclic ADP-Ribohydrolasel (human CD38 protein, Uniprot No.: P28907) as CD38 template, optionally, various tags such as His tag or Fc were fused onto CD38 protein. The antigens and proteins for detection used in the present disclosure were obtained by a process comprising: cloning into pTT5 vector (Biovector, Cat #: 102762) or pTargeT vector (Promega, A1410) respectively, transiently expressing in 293 cells or stably expressing in CHO-S cells, and purification.

Fusion protein of CD38 extracellular domain and
mouse IgG2a-Fc: CD38-ECD-mFc, used as an immunogen;
(SEQ ID No: 1)
VPRWRQQWSGPGTTKRFPETVLARCVKYTEIHPEMRHVDCQSVWDAFKGAF
ISKHPCNITEEDYQPLMKLGTQTVPCNKILLWSRIKDLAHQFTQVQRDMFT
LEDTLLGYLADDLTWCGEFNTSKINYQSCPDWRKDCSNNPVSVFWKTVSRR
FAEAACDVVHVMLNGSRSKIFDKNSTFGSVEVHNLQPEKVQTLEAWVIHGG
REDSRDLCQDPTIKELESIISKRNIQFSCKNIYRPDKFLQCVKNPEDSSCT
SEIEPRGPTIKPCPPCKCPAPNLLGGPSVFIFPPKIKDVLMISLSPIVTCV
VVDVSEDDPDVQISWFVNNVEVHTAQTQTHREDYNSTLRVVSALPIQHQDW
MSGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEKK
NWVERNSYSCSVVHEGLHNHHTTKSFSRTPGK
Note:
The underlined part represents mouse IgG2a-Fc part;
Fusion protein of CD38 extracellular domain and
human IgG1Fc: CD38-ECD-Fc, used as an immunogen;
(SEQ ID No: 2)
VPRWRQQWSGPGTTKRFPETVLARCVKYTEIHPEMRHVDCQSVWDAFKGAF
ISKHPCNITEEDYQPLMKLGTQTVPCNKILLWSRIKDLAHQFTQVQRDMFT
LEDTLLGYLADDLTWCGEFNTSKINYQSCPDWRKDCSNNPVSVFWKTVSRR
FAEAACDVVHVMLNGSRSKIFDKNSTFGSVEVHNLQPEKVQTLEAWVIHGG
REDSRDLCQDPTIKELESIISKRNIQFSCKNIYRPDKFLQCVKNPEDSSCT
SEIEPKSSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSR
WQQGNVFSCSVMHEALHNHYTQKSLSLSPGK
Note:
The underlined part represents human IgG1-Fc part;
Fusion protein of CD38 extracellular domain with
His tag: CD38-ECD-His, for use as an immunogen or
detection reagent:
(SEQ ID No: 57)
VPRWRQQWSGPGTTKRFPETVLARCVKYTEIHPEMRHVDCQSVWDAFKGAF
ISKHPCNITEEDYQPLMKLGTQTVPCNKILLWSRIKDLAHQFTQVQRDMFT
LEDTLLGYLADDLTWCGEFNTSKINYQSCPDWRKDCSNNPVSVFWKTVSRR
FAEAACDVVHVMLNGSRSKIFDKNSTFGSVEVHNLQPEKVQTLEAWVIHGG
REDSRDLCQDPTIKELESIISKRNIQFSCKNIYRPDKFLQCVKNPEDSSCT
SEIHHHHHH
Note:
The underlined part represents 6 × His tag.

### Example 2. Purification of CD38-Related Recombinant Protein

### 1. Steps for Purifying the Recombinant Protein With His Tag

The sample of cell expression supernatant was centrifuged at high speed to remove impurities, the buffer was substituted for PBS, and imidazole was added to a final concentration of 5 mM. The nickel column was equilibrated with PBS solution containing 5 mM imidazole, and washed with 2-5 times column volume. The substituted cell supernatant sample was applied onto Ni Sepharose excel column (GE, 17-3712-02). The column was washed with PBS solution containing 5 mM imidazole until the A280 reading dropped to the baseline. The chromatography column was rinsed with PBS+10 mM imidazole to remove non-specifically bound protein impurities, and the effluent was collected. The target protein was eluted with PBS solution containing 300 mM imidazole, and the elution peak was collected. The collected eluate was concentrated and further purified by gel chromatography Superdex200 (GE, 28-9893-35) with PBS as the mobile phase. The aggregate peak was removed and the main peak was collected. The resulting protein was identified by electrophoresis, peptide map and LC-MS, and the confirmed proteins were aliquoted for use. His-tagged CD38-ECD-His (SEQ ID No: 57) was obtained, for use as an immunogen for preparing the antibodies of the present disclosure or as detection reagent. CD38-ECD-His was coupled to KLH by in vitro chemistry and was used as immunogen to stimulate mouse immunity.

### 2. Steps for Purifying CD38-ECD-Fc Fusion Protein

The sample of cell expression supernatant was centrifuged at high speed to remove impurities, and the supernatant was subjected to MabSelect Sure (GE, 17-5438-01) affinity chromatography. The Mab Select Sure column was regenerated first with 0.1M NaOH, washed with pure water and then equilibrated with PBS, after the supernatant was bound, PBS was used to wash until the A280 value dropped to the baseline. The target protein was eluted with 0.1M acetic acid buffer, pH 3.5, and neutralized with 1M Tris-HCl. The eluted sample was properly concentrated, and then was further purified by PBS-equilibrated gel chromatography Superdex200 (GE, 28-9893-35), several tubes of the target proteins were pooled and concentrated to an appropriate concentration. This method was used to purify CD38-ECD-Fc (SEQ ID No: 2) fusion protein. It can also be used to purify the humanized antibody proteins in the present disclosure.

### Example 3. Obtaining and Preparation of Anti-Human CD38 Hybridoma Monoclonal Antibody

### 1. Immunization

Anti-human CD38 monoclonal antibodies were produced by immunizing mice. SJL white mice, female, 4-6 weeks old were used for experiment (Beijing Charles River Experimental Animal Technology Co., Ltd., animal production license number: SOCK (Beijing) 2012-0001).

Feeding environment: SPF grade. After the mice were purchased, they were adapted to the laboratory environment for 1 week, 12/12 hours light/dark cycle, at temperature of 20 to 25° C.; humidity of 40 to 60%. The mice that have adapted to the environment were immunized according to various protocols, 3-5 mice in each group.

The immune antigen can be CD38-ECD-His, CD38-ECD-Fc, CD38-FL-CHOS (CHOS cells transfected with human full-length of CD38), and the like. The immunization was performed with either a single reagent in combination with different immune adjuvants, or with different types of immunogens for purpose of cross-immunization The immunized site was either intraperitoneal or subcutaneous on the back, alternatively, immunization was performed alternatively on both sites. Exemplary immunization method was, for example, immunization with Titermax (Sigma Lot Num: T2684) or alum (Thremo Lot Num: 77161). The ratio of antigen to adjuvant (titermax) was 1:1, and the ratio of antigen to adjuvant (alum) was 4:1, 25-50 μg or 1×10⁷ cells/mouse (primary immunization), 25-50 μg or 1×10⁷ cells/mouse (booster immunization). On day 0, the antigen was injected intraperitoneally (IP) or subcutaneously (SC), and the immunization was repeated every two weeks after the primary immunization. Blood samples were collected every three weeks, and the antibody titer in mouse serum was determined by ELISA method. After 8 to 12 immunizations, mice with a high serum antibody titer reaching to the plateau were selected for splenocyte fusion. Three days before the splenocyte fusion, antigen solution prepared with saline was intraperitoneally injected (IP), with 25-50 μg/mouse or 1×10⁷ cells/mouse, for booster immunization.

### 2. Cell Fusion

Mice with a high serum antibody titer (see Test Example 1, ELISA method for CD38-binding) reaching to the plateau were selected for splenocyte fusion, and the selected mice were subjected to booster immunization 3 days before fusion. Hybridoma cells were obtained by fusing splenic lymphocytes with myeloma Sp2/0 cells (ATCC® CRL8287™) using an optimized PEG-mediated fusion procedure. The fused hybridoma cells were suspended in HAT complete medium (RPMI-1640 medium containing 20% FBS, 1×HAT and 1×OPI), and aliquoted into 96-well cell culture plate (1×10 ⁵ cells/150 μl/well), and incubated at 37° C., 5% CO₂. On day 5 after fusion, HAT complete medium was added, 50 μl/well, and incubated at 37° C., 5% CO₂. From day 7 to day 8 after fusion, the medium was completely changed with HT complete medium (RPMI-1640 medium containing 20% FBS, 1×HT and 1×OPI), 200 μl/well, according to the cell growth density, and incubated at 37° C., 5% CO₂.

### 3. Screening of Hybridoma Cells

10-11 days after fusion, CD38 binding ELISA assay was performed according to the growth density of the cells (see Test Example 1). The cell supernatant of positive wells detected by ELISA was tested by FACS method for the binding of CD38-FL-CHO-S (see Test Example 2). The medium in the positive wells were changed, and the cells were expanded in 24-well plates according to the density of the cells. The cell lines transferred into a 24-well plate were tested again for confirmation, and then sub-cloned for the first time. After screening the first sub-cloned cells (see Test examples 1 and 2), positive cells were preserved and subjected to the second sub-cloning. The positive cells screened in the second sub-cloning (see Test examples 1 and 2) were preserved, and used for protein expression. Hybridoma cells with high affinity to CD38 were obtained after several fusions.

The hybridoma clones m009, m011 and m160 were obtained by screening via blocking assay and binding assay. The antibodies were further prepared by serum-free cell culture method. The antibodies were purified according to the example of purification, and used for the Test Examples.

The sequence of the murine antibody variable region of hybridoma clone 009 is shown as follows:

>m009 VH: m009 heavy chain variable region sequence
SEQ ID No: 3
EFQLQQSGPELVKPGASVKISCKASGYSFTDYNLNWVKQSNGKSLEWIGVI
NPKYDAINYNQKFKDKATLTVDQSSSTAYMQLSSLTSEDSAVYYCAREGWG
KALDYWGPGTSVIVSS;
>m009 mVL: m009 light chain variable region
sequence
SEQ ID No: 4
DFVLTQSPATLSVTPGDSVSLSCRASQSIYTNLHWYQQKSHESPRLLIKYA
SQSISGIPSRFSGSGSGTDFTLSINSVETEDSGMYFCQQSNSWPLTFGAGT
KLELK;
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

The murine antibody variable region sequence of hybridoma clone m011 is as follows:

>m011 VH: m011 heavy chain variable region sequence
SEQ ID No: 5
EVQLVESGGGLVKPGGSLKLSCAASGFTFSDYGMHWVRQAPEKGLEWVAFI
SGSSSIYSYADTVKGRFTISRDNAKNTLFLQMTSLRSEDTAMYSCARNYVS
SYGYFDYWGQGTTLTVSS;
>m011 VL: m011 light chain variable region sequence
SEQ ID No: 5
DIVMTQSPASLAVSLGQRATISCRASENVDNYGISFMHWYQQKPGQPPKLL
IYRASNLESGIPARFSGSGSRTDFTLTINPVETDDVATYYCQQSNKDPLTF
GSGTKLEIK;
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

The murine antibody variable region sequence of hybridoma clone m160 is as follows:

>m160 VH: m160 heavy chain variable region sequence
SEQ ID No: 7
EVQLVESGGGLVKPGGSLKLSCVASGFTFSDYGMHWVRQAPEKGLEWIAFI
STGSSNIYYVDKVKGRFTISRDNAKNTLFLQMTSLRSEDTAIVIYYCARNY
VSSYGYFDYWGQGTTLTVSS;
>m160 VL: m160 light chain variable region sequence
SEQ ID No: 8
DIVLTQSPASLAVSLGQRATVSCRASESVDNYGISFMHWYQQKPGQPPKLL
IYRASNLESGIPARFSGSGSRTDFTLTINPVETDDVATYYCQQTNKDPLTF
GGGTKLELK;
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

The sequence of each heavy chain and light chain CDR region is shown in Table 3:

TABLE 3
Sequences of heavy and light chain
CDR regions

| Antibody | Heavy chain | | Light chain | |
|---|---|---|---|---|
| m009 | HCDR1 | DYNLN | LCDR1 | RASQSIYTNLH |
| | | SEQ ID No: 9 | | SEQ ID No: 12 |
| | HCDR2 | VINPKYDAI | LCDR2 | YASQSIS |
| | | NYNQKFKD | | SEQ ID No: 13 |
| | | SEQ ID No: 10 | | |
| | HCDR3 | EGWGKALDY | LCDR3 | QQSNSWPLT |
| | | SEQ ID No: 11 | | SEQ ID No: 14 |
| m011 | HCDR1 | DYGMH | LCDR1 | RASENVDNY |
| | | SEQ ID No: 15 | | GISFMH |
| | | | | SEQ ID No: 18 |
| | HCDR2 | FISSGSSSI | LCDR2 | RASNLES |
| | | YYADTVKG | | SEQ ID No: 19 |
| | | SEQ ID No: 16 | | |
| | HCDR3 | NYVSSYGYFDY | LCDR3 | QQSNKDPLT |
| | | SEQ ID No: 17 | | SEQ ID No: 20 |
| m160 | HCDR1 | DYGMH | LCDR1 | RASESVDNY |
| | | SEQ ID No: 15 | | GISFMH |
| | | | | SEQ ID No: 22 |
| | HCDR2 | FISTGSSNIYY | LCDR2 | RASNLES |
| | | VDKVKG | | SEQ ID No: 19 |
| | | SEQ ID No: 21 | | |
| | HCDR3 | NYVSSYGYFDY | LCDR3 | QQTNKDPLT |
| | | SEQ ID No: 17 | | SEQ ID No: 23 |

### 4. Preparation of Human IgG1 Chimeric Antibody

The candidate molecules obtained from hybridoma screening were amplified and sequenced to obtain the gene sequences encoding the variable regions. Forward and reverse primers were designed on the basis of the sequences obtained by sequencing, the genes sequenced were used as templates to construct the VH/VK gene fragment of each antibody via PCR, and then inserted into the expression vector pHr (with a signal peptide and hIgG1/hkappa constant region gene (CH1-Fc/CL) fragment) via homologous recombination to construct an expression plasmid for the expression of a full-length of recombinant chimeric antibody VH-CH1-Fc-pHr/VL-CL-pHr, resulting in chimeric antibodies ch-009, ch-011 and ch-160 of hybridoma clones m009, m011 and m160.

### Example 4. Humanization of Anti-CD38 Hybridoma Monoclonal Antibodies

The heavy/light chain variable region germline genes with high homology to m009, m011 and m160 respectively were selected as templates by aligning the IMGT human antibody heavy and light chain variable region germline gene database using MOE software analysis. The CDRs of the three murine antibodies were grafted into the corresponding human template to form a humanized antibody variable region sequence in the order of FR1-CDR1-FR2-CDR2-FR3 -CDR3 -FR4.

Selection of human FR regions and amino acid back-mutations on FR regions: Based on the resulting typical structure of murine antibody VH/VL CDR, homologous sequences of the light chain variable region (VL) and heavy chain variable region (VH) were retrieved from human germline database, arranged by FR homology from high to low, and the germline with the highest FR homology was selected as the main template; The CDR regions of the murine antibody were grafted onto the human template; Further, back-mutations designs were performed on the embedded residues, the residues that directly interact with CDR regions, and the residues that have an important influence on the conformation of VL and VH using software on the basis of the three-dimensional structure of the murine antibody; The chemically unstable amino acid residues were optimized, and the final humanized molecules were obtained.

### 1. Selection of Frameworks for Humanized Hybridoma Clone m009

For the murine antibody m009, the humanized light chain templates were IGKV3-11*01 and hJK4.1, and the humanized heavy chain templates were IGHV1-3*01 and hJH4.1. The CDRs of m009 were grafted onto the human templates and the resulting humanized variable region sequences are as follows:

>h009VH-CDR graft
SEQ ID No: 24
EVQLVQSGAEVKKPGASVKVSCKASGYTFTDYNLNWVRQAPGQRLEWMGVI
NPKYDAINYNQKFKDRVTITRDTSASTAYMELSSLRSEDTAVYYCAREGWG
KALDYWGQGTLVTVSS;
>H009VL-CDR graft
SEQ ID No: 25
EIVLTQSPATLSLSPGERATLSCRASQSIYTNLHWYQQKPGQAPRLLIYYA
SQSISGIPARFSGSGSGTDFTLTISSLEPEDFAVYYCQQSNSWPLTFGGGT
KVEIK;
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

### 2. The Back-Mutations Designed for the Humanization of Hybridoma Clone m009 are Shown in Table 4

TABLE 4
Back-mutations for the humanization of hybridoma clone m009

| VL | | VH | |
|---|---|---|---|
| h009 VL1 | graft | h009 VH1 | graft |
| h009 VL2 | A43S, Y49K | h009 VH2 | V2F, R44S, R71V |
| h009 VL3 | I2F, A43S, | h009 VH3 | V2F, R44S, M48I, V67A, R71V |
| | Y49K, Y87F | h009 VH4 | V2F, R38K, R44S, R66K, |
| | | | I69L, R71V, T73Q |
| | | h009 VH5 | V2F, R38K, R44S, M48I, V67A, |
| | | | R66K, I69L, R71V, T73Q |

Note:
Graft means that CDRs of the murine antibodies were grafted onto the human germline FR regions; “A43S” means the “A” at position 43 (numbered according to the Kabat Numbering criteria) was back-mutated to “S”, and so on.

The humanized antibody variable region sequences of the hybridoma clone m009 are as follows:

>h009 VH1
(SEQ ID No: 24)
EVQLVQSGAEVKKPGASVKVSCKASGYTFTDYNLNWVRQAPGQRLEWMGVI
NPKYDAINYNQKFKDRVTITRDTSASTAYMELSSLRSEDTAVYYCAREGWG
KALDYWGQGTLVTVSS
>h009 VH2
(SEQ ID No: 26)
E [symbol] QLVQSGAEVKKPGASVKVSCKASGYTFTDYNLNWVRQAPGQ [symbol] LEWMGVI
NPKYDAINYNQKFKDRVTIT [symbol] DTSASTAYMELSSLRSEDTAVYYCAREGWG
KALDYWGQGTLVTVSS
>h009 VH3
(SEQ ID No: 27)
EFQLVQSGAEVKKPGASVKVSCKASGYTFTDYNLNWVRQAPGQSLEWIGVI
NPKYDAINYNQKFKDRATITVDTSASTAYMELSSLRSEDTAVYYCAREGWG
KALDYWGQGTLVTVSS
>h009 VH4
(SEQ ID No: 28)
EFQLVQSGAEVKKPGASVKVSCKASGYTFTDYNLNWVKQAPGQSLEWMGVI
NPKYDAINYNQKFKDKVTLTVDQSASTAYMELSSLRSEDTAVYYCAREGWG
KALDYWGQGTLVTVSS
>h009 VH5
(SEQ ID No: 29)
EFQLVQSGAEVKKPGASVKVSCKASGYTFTDYNLNWVKQAPGQSLEWIGVI
NPKYDAINYNQKFKDKATLTVDQSASTAYMELSSLRSEDTAVYYCAREGWG
KALDYWGQGTLVTVSS
>h009 VL1
(SEQ ID No: 25)
EIVLTQSPATLSLSPGERATLSCRASQSIYTNLHWYQQKPGQAPRLLIYYA
SQSISGIPARFSGSGSGTDFTLTISSLEPEDFAVYYCQQSNSWPLTFGGGT
KVEIK
>h009 VL2
(SEQ ID No: 30)
EIVLTQSPATLSLSPGERATLSCRASQSIYTNLHWYQQKPGQSPRLLIKYA
SQSISGIPARFSGSGSGTDFTLTISSLEPEDFAVYYCQQSNSWPLTFGGGT
KVEIK
>h009 VL3
(SEQ ID No: 31)
EFVLTQSPATLSLSPGERATLSCRASQSIYTNLHWYQQKPGQSPRLLIKYA
SQSISGIPARFSGSGSGTDFTLTISSLEPEDFAVYFCQQSNSWPLTFGGGT
KVEIK.

The humanized light chain variable region and heavy chain variable region described above were respectively combined with human germline light chain constant region (such as human κ, λ chain light chain constant regions) and heavy chain constant region (such as the heavy chain constant region of human IgG1, IgG2, IgG3, or IgG4 or variant thereof), to form a heavy chain and light chain of the humanized antibody, thereby resulting in a complete humanized antibody of m009 (h009). As an example, full-length humanized antibodies (h009-01 to h009-15) were obtained by combining the above-mentioned h009 antibody heavy chain variable region and light chain variable region with the human IgG1 heavy chain constant region as shown in SEQ ID No: 43 and the human kappa light chain constant region as shown in SEQ ID No: 45 respectively. The variable region sequences are shown in Table 5:

TABLE 5
Heavy chain variable region and light chain variable
sequences of humanized antibody h009

| Variable region | h009 VL1 | h009 VL2 | h009 VL3 |
|---|---|---|---|
| h009 VH1 | h009-01 | h009-06 | h009-11 |
| h009 VH2 | h009-02 | h009-07 | h009-12 |
| h009 VH3 | h009-03 | h009-08 | h009-13 |
| h009 VH4 | h009-04 | h009-09 | h009-14 |
| h009 VH5 | h009-05 | h009-10 | h009-15 |

Note:
For example, for “h009-07” in the table, it suggests that the heavy and light chain variable region of the humanized antibody h009-07 are h009 VH2 and h009VL2 respectively, and so on.

### 3. Selection of Frameworks for Humanized Hybridoma Clone m011

For the murine antibody m011, the humanized light chain templates were IGKV4-1*01 and hJK4.1, and the humanized heavy chain templates were IGHV3-7*01 and hJH6.1. In order to eliminate potential hot spots, N 82A T (according to the Kabat Numbering criteria, asparagine (abbr. N or Asn) on position 82A was replaced with threonine (abbr. T or Thr)) and N76S (according to the Kabat Numbering criteria, asparagine (abbr. N or Asn) on position 76 was replaced with serine (abbr. S or Ser)) were introduced into the FR regions of human germline IGHV3-7*01 and hJH6.1; the CDRs of m011 were grafted onto human templates and the resulting humanized variable region sequences are as follows:

>h011VH-CDR graft
SEQ ID No: 32
EVQLVESGGGLVQPGGSLRLSCAASGFTESDYGMHWVRQAPGKGLEWVAFI
SSGSSSIYYADTVKGRFTISRDNAKSSLYLQMTSLRAEDTAVYYCARNYVS
SYGYFDYWGQGTTVTVSS;
>h011VL-CDR graft
SEQ ID No: 33
DIVMTQSPDSLAVSLGERATINCRASENVDNYGISFMHWYQQKPGQPPKLL
IYRASNLESGVPDRFSGSGSGTDFTLTISSLQAEDVAVYYCQQSNKDPLTF
GGGTKVEIK;
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

### 4. The Back-Mutations Designed for the Hybridoma Clone m011 are Shown in Table 6

TABLE 6
Back-mutations for the humanization of hybridoma clone m011

| VL | | VH | |
|---|---|---|---|
| h011 VL1 | graft | h011 VH1 | graft, N 82A T, N76S |
| h011 VL2 | G68R | h011 VH2 | Y79F, N 82A T, |
| h011 VL3 | V58I, G68R, | | Y91S, N76S |
| | V85T | | |

Note:
graft means that the murine antibody CDRs were grafted onto the human germline FR region sequences. G68R means that “G” at position 68 (numbered according to the Kabat Numbering criteria) was back-mutated to R after grafting, and so on.

The specific sequences of the variable regions of the h011 humanized antibody are as follows:

>h011 VH1
(SEQ ID No: 32)
EVQLVESGGGLVQPGGSLRLSCAASGFTFSDYGMHWVRQAPGKGLEWVAFI
SSGSSSIYYADTVKGRFTISRDNAKSSLYLQMTSLRAEDTAVYYCARNYVS
SYGYFDYWGQGTTVTVSS
>h011 VH2
(SEQ ID No: 34)
EVQLVESGGGLVQPGGSLRLSCAASGFTFSDYGMHWVRQAPGKGLEWVAFI
SSGSSSIYYADTVKGRFTISRDNAKSSLFLQMTSLRAEDTAVYSCARNYVS
SYGYFDYWGQGTTVTVSS
>h011 VL1
(SEQ ID No: 33)
DIVMTQSPDSLAVSLGERATINCRASENVDNYGISFMHWYQQKPGQPPKLL
IYRASNLESGVPDRFSGSGSGTDFTLTISSLQAEDVAVYYCQQSNKDPLTF
GGGTKVEIK
>h011 VL2
(SEQ ID No: 35)
DIVMTQSPDSLAVSLGERATINCRASENVDNYGISFMHWYQQKPGQPPKLL
IYRASNLESGVPDRFSGSGSRTDFTLTISSLQAEDVAVYYCQQSNKDPLTF
GGGTKVEIK
>h011VL3
(SEQ ID No: 36)
DIVMTQSPDSLAVSLGERATINCRASENVDNYGISFMHWYQQKPGQPPKLL
IYRASNLESGIPDRFSGSGSRTDFTLTISSLQAEDVATYYCQQSNKDPLTF
GGGTKVEIK.

The humanized light chain variable region and heavy chain variable region described above were respectively combined with human germline light chain constant region (such as human κ, λ chain light chain constant regions) and heavy chain constant regions (such as the heavy chain constant region of human IgG1, IgG2, IgG3, or IgG4 or variant thereof), to form a heavy chain and light chain of the humanized antibody, thereby resulting in a complete humanized antibody of m011 (h011). As an example, full-length humanized antibodies (h011-01 to h011-06) were obtained by combining the above-mentioned h011 antibody heavy chain variable region and light chain variable region with the human IgG1 heavy chain constant region as shown in SEQ ID No: 43 and the human kappa light chain constant region as shown in SEQ ID No: 45 respectively. The variable region sequences are shown in Table 7:

TABLE 7
Combinations of heavy and light chain variable regions
of humanized antibody h0011

| | h011 VL1 | h011 VL2 | h011 VL3 |
|---|---|---|---|
| h011 VH1 | h011-01 | h011-03 | h011-05 |
| h011 VH2 | h011-02 | h011-04 | h011-06 |

Note:
For example, for “h011-04” in the table, it suggests that the heavy and light chain variable region of the humanized antibody h011-04 are h011 VH2 and h011VL2 respectively, and so on.

### 5. Selection of Frameworks for Humanized Hybridoma Clone m160

For the murine antibody m160, the humanized light chain templates were IGKV4-1*01 and hJK4.1 and the humanized heavy chain templates were IGHV3-7*01 and hJH6.1. In order to eliminate potential hot spots present in the human germline FR regions, mutations S77T (according to the Kabat Numbering criteria, serine (abbr. S or Ser) on position 77 was replaced with threonine (Abbr. T or Thr)) and N 82A T (according to the Kabat Numbering criteria, asparagine (abbr. N or Asn) on position 82A was replaced with threonine (abbr. T or Thr)) were introduced into the FR regions of human germline IGHV3-7*01 and hJH6.1. The m160 CDRs were grafted onto the human template, and the resulting humanized variable region sequences are as follows:

>h160VH-CDR graft
SEQ ID No: 37
EVQLVESGGGLVQPGGSLRLSCAASGFTFSDYGMHWVRQAPGKGLEWVAFI
STGSSNIYYVDKVKGRFTISRDNAKNTLYLQMTSLRAEDTAVYYCARNYVS
SYGYFDYWGQGTTVTVSS
>h160VL-CDR graft
SEQ ID No: 38
DIVMTQSPASLAVSLGERATINCRASESVDNYGISFMHWYQQKPGQPPKLL
IYRASNLESGVPDRFSGSGSGTDFTLTISSLQAEDVAVYYCQQTNKDPLTF
GGGTKVEIK
Note:
The CDR sequences determined according to Kabat
Numbering Criteria are underlined, the FR sequences
are presented in italic, and the sequences are
arranged in the order of FR1-CDR1-FR2-CDR2-FR3-
CDR3-FR4.

### 6. The Back-Mutations Designed for the Humanization of Hybridoma Clone m160 are Shown in Table 8

TABLE 8
Back-mutations for the humanization of hybridoma clone m160

| VL | | VH | |
|---|---|---|---|
| h160 VL1 | Graft, D9A | h160 VH1 | Graft, S77T, N 82A T |
| h160 VL2 | M4L, D9A | h160 VH2 | V48I, S77T, N 82A T |
| h160 VL3 | M4L, D9A, | | |
| | D60A, G68R | | |
| h160 VL4 | M4L, D9A, N22S, | | |
| | V58I, D60A, G68R | | |

Note:
Graft means that CDRs of the murine antibodies were grafted onto the human germline FR regions; “M4L″ means the “M” on position 4 (numbered according to the Kabat Numbering criteria) was back-mutated to “L” after grafting, and so on.

The specific sequences of the variable regions of the humanized antibody h160 are as follows:

>h160 VH1
(SEQ ID No: 37)
EVQLVESGGGLVQPGGSLRLSCAASGFTFSDYGMHWVRQAPGKGLEWVAFI
STGSSNIYYVDKVKGRFTISRDNAKNTLYLQMTSLRAEDTAVYYCARNYVS
SYGYFDYWGQGTTVTVSS;
>h160 VH2
(SEQ ID No: 39)
EVQLVESGGGLVQPGGSLRLSCAASGFTFSDYGMHWVRQAPGKGLEWIAFI
STGSSNIYYVDKVKGRFTISRDNAKNTLYLQMTSLRAEDTAVYYCARNYVS
SYGYFDYWGQGTTVTVSS;
>h160 VL1
(SEQ ID No: 38)
DIVMTQSPASLAVSLGERATINCRASESVDNYGISFMHWYQQKPGQPPKLL
IYRASNLESGVPDRFSGSGSGTDFTLTIS SLQAEDVAVYYCQQTNKDPLT
FGGGTKVEIK;
>h160 VL2
(SEQ ID No: 40)
DIVLTQSPASLAVSLGERATINCRASESVDNYGISFMHWYQQKPGQPPKWY
RASNLESGVPDRFSGSGSGTDFTLTISSLQAEDVAVYYCQQTNKDPLTFGG
GTKVEIK;
>h160 VL3
(SEQ ID No: 41)
DIVLTQSPASLAVSLGERATINCRASESVDNYGISFMHWYQQKPGQPPKWY
RASNLESGVPARFSGSGSRTDFTLTISSLQAEDVAVYYCQQTNKDPLTFGG
GTKVEIK;
>h160 VL4
(SEQ ID No: 42)
DIVLTQSPASLAVSLGERATISCRASESVDNYGISFMHWYQQKPGQPPKLL
IYRASNLESGIPARFSGSGSRTDFTLTISSLQAEDVAVYYCQQTNKDPLTF
GGGTKVEIK.

The humanized light chain variable region and heavy chain variable region described above were respectively combined with human germline light chain constant region (such as human κ, λ chain light chain constant regions) and heavy chain constant regions (such as the heavy chain constant region of human IgG1, IgG2, IgG3, or IgG4 or variant thereof), to form a heavy chain and light chain of the humanized antibody, thereby resulting in a complete humanized antibody of m160 (h160). As an example, full-length humanized antibodies (h160-01 to h160-08) were obtained by combining the above-mentioned h160 antibody heavy chain variable region and light chain variable region with the human IgG1 heavy chain constant region as shown in SEQ ID No: 43 and the human kappa light chain constant region as shown in SEQ ID No: 45 respectively. The variable region sequences are shown in Table 9:

TABLE 9
Heavy chain variable region and light chain variable sequences of
humanized antibody h016

| | h160 VL1 | h160 VL2 | h160 VL3 | h160 VL4 |
|---|---|---|---|---|
| h160 VH1 | h160-01 | h160-02 | h160-03 | h160-04 |
| h160 VH2 | h160-05 | h160-06 | h160-07 | h160-08 |

Note:
For example, for “h160-07” in the table, it suggests that the heavy and light chain variable region of the humanized antibody h160-07 are h160 VH2 and h160 VL3 respectively, and so on.

### Example 5. Construction and Expression of Anti-Human CD38 Humanized Antibody IgG1 or IgG1-E333A Format

Various primers were designed, VH/VK gene fragment of each humanized antibody was amplified by PCR and then inserted into the expression vector pHr (with a signal peptide and constant region gene (CH1-FC/CL) fragment, constructed in laboratory) via homologous recombination to construct an expression vector for a full-length antibody VH-CH1-FC-pHr/VK-CL-pHr. For the humanized antibody, the light chain constant region may be selected from human κ or λ chain light chain constant region, and the heavy chain constant region may be selected from the heavy chain constant region of human IgG1, IgG2, IgG3, or IgG4 or variant thereof. Non-limiting examples include optimizing the constant region of human IgG1, IgG2 or IgG4 to improve antibody's function. For example, the IgG1-E333A constant region can be obtained by introducing E333A point-mutation into IgG1, which can enhance the binding ability of IgG1-Fc to C1q and consequently enhance the CDC function of the antibody (see U.S. Pat. No. 6,528,624). The following specific light/heavy chain constant regions are not intended to limit the antibody constant regions of the present disclosure, and other antibody light/heavy chain constant regions and variants thereof known in the art can also be used.

Exemplary heavy and light chain constant regions are as follows:

IgG1 heavy chain constant region:
SEQ ID No: 43
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVF
SCSVMHEALHNHYTQKSLSLSPGK;
IgG1-E333A heavy chain constant region:
SEQ ID No: 44
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIAKTISKAKGQPREPQVYTLPPSRDELTKNQVSLTCLVKGF
YPSDIAVEWESNGQPENNYKTTPPVLDSDGSFFLYSKLTVDKSRWQQGNVF
SCSVMHEALHNHYTQKSLSLSPGK;
kappa light chain constant region:
SEQ ID No: 45
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSF
NRGEC.

As an example, the humanized light chain variable region and heavy chain variable region of the above-mentioned hybridoma clones m009, m011, and m160 were respectively combined with the human IgG1 heavy chain constant region as shown in SEQ ID No: 43 and the human kappa light chain constant region as shown in SEQ ID No: 45, and the resulting full-length humanized antibodies are shown in Table 5, Table 7 and Table 9; As another example, the humanized light chain variable region and heavy chain variable region of the above-mentioned hybridoma clones m009, m011, and m160 were respectively combined with the human IgG1-E333A heavy chain constant region as shown in SEQ ID No: 44 and the human kappa light chain constant region as shown in SEQ ID No: 45, and the resulting full-length humanized antibodies are shown in Table 10:

TABLE 10
Variable region sequences of the humanized antibodies (heavy
chain constant region is human IgG1-E333A, and light chain
constant region is kappa)

| | Heavy chain | Light chain variable |
|---|---|---|
| Antibody | variable region VH | region VL |
| h009-01E | h009 VH1 | h009 VL1 |
| h009-02E | h009 VH2 | h009 VL1 |
| h009-03E | h009 VH3 | h009 VL1 |
| h009-04E | h009 VH4 | h009 VL1 |
| h009-05E | h009 VH5 | h009 VL1 |
| h009-06E | h009 VH1 | h009 VL2 |
| h009-07E | h009 VH2 | h009 VL2 |
| (Also referred | | |
| to as hu9E) | | |
| h009-08E | h009 VH3 | h009 VL2 |
| h009-09E | h009 VH4 | h009 VL2 |
| h009-10E | h009 VH5 | h009 VL2 |
| h009-11E | h009 VH1 | h009 VL3 |
| h009-12E | h009 VH2 | h009 VL3 |
| h009-13E | h009 VH3 | h009 VL3 |
| h009-14E | h009 VH4 | h009 VL3 |
| h009-15E | h009 VH5 | h009 VL3 |
| h011-01E | h011 VH1 | h011 VL1 |
| (Also referred | | |
| to as hu11E) | | |
| h011-02E | h011 VH2 | h011 VL2 |
| h011-03E | h011 VH1 | h011 VL3 |
| h011-04E | h011 VH2 | h011 VL1 |
| h011-05E | h011 VH1 | h011 VL2 |
| h011-06E | h011 VH2 | h011 VL3 |
| h160-01E | h160 VH1 | h160 VL1 |
| (Also referred | | |
| to as hu160E) | | |
| h160-02E | h160 VH1 | h160 VL2 |
| h160-03E | h160 VH1 | h160 VL3 |
| h160-04E | h160 VH1 | h160 VL4 |
| h160-05E | h160 VH2 | h160 VL1 |
| h160-06E | h160 VH2 | h160 VL2 |
| h160-07E | h160 VH2 | h160 VL3 |
| h160-08E | h160 VH2 | h160 VL4 |

As as example, the full-length amino acid sequences of humanized antibodies h009-07, hu9E, h011-01, hu11E, h160-01 and hu160E are as follows:

Heavy chain sequence of the antibody h009-07:
SEQ ID No: 46
EFQLVQSGAEVKKPGASVKVSCKASGYTFTDYNLNWVRQAPGQSLEWMGVI
NPKYDAINYNQKFKDRVTITVDTSASTAYMELSSLRSEDTAVYYCAREGWG
KALDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLY
SKLTVDKSRWQQGNVFSCSVMHEALHNHYTQKSLSLSPGK;
Light chain sequence of the antibody h009-07:
SEQ ID No: 47
EIVLTQSPATLSLSPGERATLSCRASQSIYTNLHWYQQKPGQSPRLLIKYA
SQSISGIPARFSGSGSGTDFTLTISSLEPEDFAVYYCQQSNSWPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC;
Heavy chain sequence of the antibody hu9E:
SEQ ID No: 48
EFQLVQSGAEVKKPGASVKVSCKASGYTFTDYNLNWVRQAPGQSLEWMGVI
NPKYDAINYNQKFKDRVTITVDTSASTAYMELSSLRSEDTAVYYCAREGWG
KALDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIAKTISKAKGQPREPQVYTLPPSRD
ELTKNQVSLTCLVKGFYPSDIAVEWESNGQPENNYKTTPPVLDSDGSFFLY
SKLTVDKSRWQQGNVFSCSVMHEALHNHYTQKSLSLSPGK;
Light chain sequence of the antibody hu9E:
SEQ ID No: 47
EIVLTQSPATLSLSPGERATLSCRASQSIYTNLHWYQQKPGQSPRLLIKYA
SQSISGIPARFSGSGSGTDFTLTISSLEPEDFAVYYCQQSNSWPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC;
Heavy chain sequence of the antibody h011-01:
SEQ ID No: 49
EVQLVESGGGLVQPGGSLRLSCAASGFTFSDYGMHWVRQAPGKGLEWVAFI
SSGSSSIYYADTVKGRFTISRDNAKSSLYLQMTSLRAEDTAVYYCARNYVS
SYGYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFF
LYSKLTVDKSRWQQGNVFSCSVMHEALHNHYTQKSLSLSPGK;
Light chain sequence of the antibody h011-01:
SEQ ID No: 50
DIVMTQSPDSLAVSLGERATINCRASENVDNYGISFMHWYQQKPGQPPKLL
IYRASNLESGVPDRFSGSGSGTDFTLTISSLQAEDVAVYYCQQSNKDPLTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC;
Heavy chain sequence of the antibody hu11E:
SEQ ID No: 51
EVQLVESGGGLVQPGGSLRLSCAASGFTFSDYGMHWVRQAPGKGLEWVAFI
SSGSSSIYYADTVKGRFTISRDNAKSSLYLQMTSLRAEDTAVYYCARNYVS
SYGYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVV
SVLTVLHQDWLNGKEYKCKVSNKALPAPIAKTISKAKGQPREPQVYTLPPS
RDELTKNQVSLTCLVKGFYPSDIAVEWESNGQPENNYKTTPPVLDSDGSFF
LYSKLTVDKSRWQQGNVFSCSVMHEALHNHYTQKSLSLSPGK;
Light chain sequence of the antibody hu11E:
SEQ ID No: 50
DIVMTQSPDSLAVSLGERATINCRASENVDNYGISFIVIHWYQQKPGQPPK
LLTYRASNLESGVPDRFSGSGSGTDFTLTISSLQAEDVAVYYCQQSNKDPL
TFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTH
QGLSSPVTKSFNRGEC;
Heavy chain sequence of the antibody h160-01:
SEQ ID No: 52
EVQLVESGGGLVQPGGSLRLSCAASGFTFSDYGMHWVRQAPGKGLEWVAFI
STGSSNIYYVDKVKGRFTISRDNAKNTLYLQMTSLRAEDTAVYYCARNYVS
SYGYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFF
LYSKLTVDKSRWQQGNVFSCSVMHEALHNHYTQKSLSLSPGK;
Light chain sequence of the antibody h160-01:
SEQ ID No: 53
DIVMTQSPASLAVSLGERATINCRASESVDNYGISFMHWYQQKPGQPPKLL
IYRASNLESGVPDRFSGSGSGTDFTLTISSLQAEDVAVYYCQQTNKDPLTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC;
Heavy chain sequence of the antibody hu160E:
SEQ ID No: 54
EVQLVESGGGLVQPGGSLRLSCAASGFTFSDYGMHWVRQAPGKGLEWVAFI
STGSSNIYYVDKVKGRFTISRDNAKNTLYLQMTSLRAEDTAVYYCARNYVS
SYGYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVV
SVLTVLHQDWLNGKEYKCKVSNKALPAPIAKTISKAKGQPREPQVYTLPPS
RDELTKNQVSLTCLVKGFYPSDIAVEWESNGQPENNYKTTPPVLDSDGSFF
LYSKLTVDKSRWQQGNVFSCSVMHEALHNHYTQKSLSLSPGK;
Light chain sequence of the antibody hu160E:
SEQ ID No: 53
DIVMTQSPASLAVSLGERATINCRASESVDNYGISFMHWYQQKPGQPPKLL
IYRASNLESGVPDRFSGSGSGTDFTLTISSLQAEDVAVYYCQQTNKDPLTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC;

The anti-CD38 antibody, Daratumumab (abbr. Dara, refer to WHO Drug Information, Vol. 24, No. 1, 2010 for sequences) was used as a control antibody in the present disclosure, and its heavy chain and light chain sequences are as follows:

Heavy chain sequence of Dara:
SEQ ID No: 55
EVQLLESGGGLVQPGGSLRLSCAVSGFTFNSFAMSWVRQAPGKGLEWVSAI
SGSGGGTYYADSVKGRFTISRDNSKNTLYLQMNSLRAEDTAVYFCAKDKIL
WFGEPVFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK;
Light chain sequence of Dara:
SEQ ID No: 56
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPPTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

The performance and effect of the present antibdoies were verified by the following tests:

### Test Example 1. CD38 Protein-Binding ELISA Test for CD38 Antibodies

The affinity of the anti-CD38 antibodies were determined by the amount of the antibodies binding to CD38 immobilized on the ELISA plate. 2 μg/ml streptavidin (Abcam, CAT #ab123480) was coated on a 96-well ELISA plate (Costar, CAT #3590), the plate was washed, blocked, and then 2 μg/ml biotin-labeled CD38-ECD-His was added. After incubation, the diluted anti-CD38 antibody samples with various concentrations were added, washed, and then added with horseradish peroxidase-goat anti-human F(ab′)₂ antibody (Jackson, CAT #109-036-097). The plate was washed again and tetramethyl benzidine solution was added for color reaction. Finally, stop solution was added. OD450 was measured on a microplate reader and EC50 value was calculated. The results are shown in Table 11.

TABLE 11
Affinity of CD38 humanized antibody

| Antibody | EC50 (μg/ml) |
|---|---|
| hu9E | 0.02594 |
| hu11E | 0.02762 |
| hu160E | 0.02801 |

The results show that the humanized antibodies of the present disclosure can specifically bind to CD38 protein with strong binding ability.

### Test Example 2. Binding Assay of CD38 Antibodies to CD38-FL-CHO-S Cells

CHO-S cells (FreeStyle™ CHO-S cells, Invitrogen, R80007) stably transfected with full-length human CD38 (Uniprot number: P28907) (CD38-FL-CHOS) were cultivated in CD CHO culture medium (Gibco, REF #10743-029). 1×10⁶ cells/ml of CD38-FL-CHO-S cells were blocked with 1% BSA, and the diluted anti-CD38 antibody samples with various concentrations were added, washed twice, and then Alexa Fluor 488-goat anti-human (H+L) antibody (Invitrogen, CAT #A11013) was added, washed twice, and the fluorescence signal values were read with flow cytometer. The results are shown in Table 12.

TABLE 12
Affinity of CD38 humanized antibody

| Antibody | EC50 (μg/ml) |
|---|---|
| hu9E | 0.4020 |
| hu11E | 0.4813 |
| hu160E | 0.4740 |

FACS test results show that the humanized antibodies of the present disclosure have strong binding ability to natural CD38 present on the cell surface.

### Test Example 3. Inhibitory Assay of CD38 Antibodies on CD38 Enzyme Activity

CD38-ECD-His was prepared to a solution with a concentration of 4 μg/ml with 20 mM Tris-HCl (pH 6.5) buffer. Similarly, various concentrations of anti-CD38 antibody samples were prepared with buffer. 25 μl of each of CD38-ECD-His and anti-CD38 antibody sample were added into a 96-well plate with a transparent bottom and black wall (Corning, CAT #3603). After incubated at room temperature for 15 minutes, 50 μl of 200 μM substrate NGD (Sigma, CAT #N5131-25MG) was added. After incubated at room temperature for 2 hours, the production of cyclic GDP ribose (cGDPR) was measured by FlexStation 3 (Molecular Devices), at emission wavelength of 410 nm (excitation light of 300 nm). The results are shown in Table 13.

TABLE 13
Inhibitory results of CD38 humanized antibody on CD38 enzyme activity

| Antibody | IC50 (μg/ml) | Imax (Maximum inhibition rate %) |
|---|---|---|
| Dara | 3.147 | 44.54 |
| hu9E | 1.559 | 89.63 |
| hu11E | 1.827 | 44.31 |
| hu160E | 1.199 | 47.67 |

The test results show that hu9E exhibits a maximum inhibition rate of 89.63% for enzyme activity, significantly superior to that of the control antibody Dara; whereas hu11E and hu160E have a maximum inhibition rates of 44.31% and 47.67% respectively for enzyme activity, comparable to that of the control antibody.

### Test Example 4. In Vitro ADCC Assay of CD38 Antibodies on Molp-8 and Daudi Cells

Human multiple myeloma cell line Molp-8 (Cobioer, Nanjing, CBP60562) or human Burkitt's lymphoma cell line Daudi cells (ATCC, CCL-123) were collected, centrifuged at 1000 rpm for 5 minutes, and suspended with phenol red-free RPMI 1640 medium (Gibco, CAT #11835-030) containing 10% ultra-low IgG Fetal Bovine Serum (Gibco, CAT #1921005PJ). The cells were counted with Cytometer (Countstar, IC1000) and diluted to 1×10⁵ cells/ml.

Peripheral blood mononuclear cells (PBMCs) were isolated from fresh human blood by Ficoll (GE, CAT #17-5442-02), re-suspended in phenol red-free RPMI 1640 medium, counted with Cytometer, and diluted to 3×10⁶ cells/ml.

50 μl of each of Molp-8 (or Daudi) and different concentrations of CD38 antibodies or negative control IgG (C25-hIgG1 (WT), prepared in the laboratory) were added into a 96-well plate at a ratio of 1:1, incubated for 30 minutes (37° C., 5% CO₂), and 50 μl of effector cell PBMCs were added at the ratio of 30:1 (effector cell:target cell). After incubated for 4 hours (37° C., 5% CO₂), the release of LDH (lactate dehydrogenase) was detected with CytoTox 96 Non-Radiocytotoxicity Test Kit (Promega, CAT #G1780). 50 μl of cell supernatant and 50 μl of CytoTox 96® Reagent were added. After incubated at room temperature for 30 minutes, stop solution was added. The absorbance values (490 nm) were detected with FlexStation 3 (Molecular Devices). The results are shown in Table 14.

TABLE 14
ADCC results of CD38 humanized antibodies on target cells

| | Molp-8 | | Daudi | |
|---|---|---|---|---|
| | | Emax | | Emax |
| | IC50 | (Maximum | IC50 | (Maximum |
| Antibody | (μg/ml) | efficiency, %) | (μg/ml) | efficiency %) |
| hu9E | 4.5 | 83.6 | 4.9 | 102.5 |
| hu11E | 3.8 | 72.0 | 2.6 | 92.5 |
| hu160E | 2.7 | 71.5 | 3.1 | 96.7 |

Experimental results show that the humanized antibodies of the present disclosure have strong ADCC effect on Molp-8 and Daudi cells in vitro, and can significantly achieve the lysis the target cells.

### Test Example 5. CDC of CD38 Antibodies on Molp-8 and Daudi Cells by In Vitro Assay

Molp-8 or Daudi cells were collected, centrifuged at 1000 rpm for 5 minutes and re-suspended. The cells were counted with Cytometer (Countstar, IC1000), and re-suspended in phenol red-free RPMI 1640 medium (Gibco, CAT #11835-030) containing 10% ultra-low IgG Fetal Bovine Serum (Gibco, CAT #1921005PJ) at 1×10⁶cells/ml. Subsequently, the cells were plated in a 96-well plate (Corning, CAT #3903) at 5×10⁴ cells/well (50 μl/well). Then, 50 μl of various concentrations of anti-CD38 antibodies and negative control were added. After incubated for 30 minutes (37° C., 5% CO₂), 50 μl of human serum (laboratory-made) was added into each well. After incubated for 2 hours (37° C., 5% CO₂), 16.6 μl of Alamar Blue Reagent (Thermo, CAT #DAL1025) was added to each well, and incubated for 20 hours (37° C., 5% CO₂). Finally, the detection was performed with FlexStation 3 (Molecular Devices) at emission wavelength of 585 nm (excitation wavelength of 570 nm), and the results are shown in Table 15.

TABLE 15
CDC results of humanized CD38 antibodies on target cells

| | Molp-8 | | Daudi | |
|---|---|---|---|---|
| | | Emax | | Emax |
| | IC50 | (Maximum | IC50 | (Maximum |
| Antibody | (μg/ml) | efficiency, %) | (μg/ml) | efficiency, %) |
| hu9E | 281.4 | 79.7 | 51.1 | 92.8 |
| hu11E | 289.9 | 67.4 | 70.1 | 91.4 |
| hu160E | 393.2 | 61.5 | 65.7 | 88.1 |

Experimental results show that the humanized antibodies of the present disclosure have strong CDC effect on Molp-8 and Daudi cells in vitro, and can significantly achieve the lysis the target cells.

### Test Example 6. In Vitro ADCP Reporting System Test of CD38 Antibodies on Molp-8 and Daudi Cells

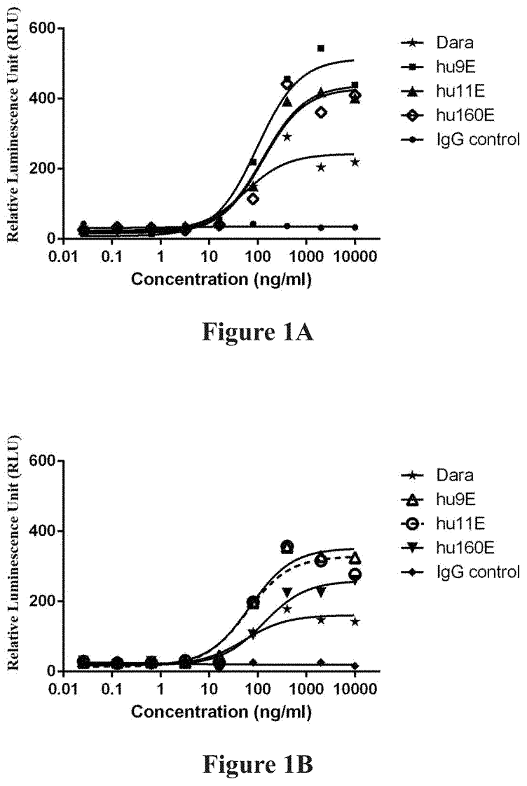

Molp-8 or Daudi cells were collected, centrifuged at 1000 rpm for 5 minutes and re-suspended. The cells were counted with Cytometer (Countstar, IC1000), and re-suspended in phenol red-free RPMI 1640 medium (Gibco, CAT #11835-030) containing 10% ultra-low IgG Fetal Bovine Serum (Gibco, CAT #1921005PJ) at 1×10⁶ cells/ml. Subsequently, the cells were plated in a 96-well plate (Corning, CAT #3903) at 2.5×10⁴ cells/well (25 μl/well), 25 μl of various concentrations of anti-CD38 antibodies and negative control were added, and incubated for 30 minutes (37° C., 5% CO₂). Jurkat (Jurkat-Lucia™ NFAT Cells, Invivogene) cells stably transformed with full-length human FcγIIa (Uniprot number: P12318) were collected as effector cells, and 7.5×10⁴ cells/well (50 μl/well) of effector cells were added into a 96-well plate incubated with the target cells and antibodies. After incubated for 6 hours (37° C., 5% CO₂), 10 μl of supernatant was transferred into a new 96-well plate (Corning, CAT #3903), 90 μl/well of QUANTI-Luc (Invivogene, rep-qlc1) was added, and the chemiluminescence was detected with VITOR (VITOR3, PerkinElmer). The results are shown in Table 16 and FIG. 1.

TABLE 16
In vitro ADCP reporter system test results of CD38
humanized antibodies on target cells

| | Signal ratio relative to Dara (fold) | |
|---|---|---|
| Antibody | Molp-8 | Daudi |
| Dara | 1.00 | 1.00 |
| hu9E | 2.00 | 2.30 |
| hu11E | 1.83 | 2.00 |
| hu160E | 1.87 | 1.80 |

The experimental results show that all the humanized antibodies of the present disclosure have in vitro ADCP reporter system test effects on Molp-8 and Daudi cells significantly better than that of the control antibody Dara.

### Test Example 7. Affinity of CD38 Antibodies Detected by BIAcore

The affinities of the chimeric antibodies (ch-009, -011 and -160 prepared according to Example 3), humanized antibodies of the present disclosure and Dara to the human CD38-ECD-His antigen were detected by Biacore instrument.

Human Fc Capture Molecule was covalently coupled to CM5 biosensing chip (CAT #BR-1005-30, GE) according to the method described in the manual of the Human Fc Capture Kit (CAT #BR-1008-39, GE), for affinity capture of the antibodies to be tested. Then the human CD38-ECD-His antigen passed through the surface of the chip, and a real-time detection for the reaction signal was performed with Biacore instrument. The resulting binding and dissociation curves were fitted to calculate the affinity values. After the dissociation of each cycle was completed in the experiment, the biochip was washed and regenerated with the regeneration solution supplied in the Human Fc Capture Kit (GE). The results are shown in Table 17 and Table 18.

TABLE 17
Results of the affinity of anti-CD38 antibody to human CD38 by BIAcore

| Antibody | ka (1/Ms) | kd (1/s) | KD (M) |
|---|---|---|---|
| ch-009 | 2.04E+06 | 4.77E−04 | 2.34E−10 |
| h009-07 | 2.11E+06 | 2.10E−03 | 9.95E−10 |
| h009-08 | 1.89E+06 | 2.23E−03 | 1.18E−09 |
| h009-09 | 2.29E+06 | 4.89E−03 | 2.14E−09 |
| h009-10 | 2.04E+06 | 4.33E−03 | 2.12E−09 |
| h009-11 | 2.33E+06 | 1.21E−02 | 5.17E−09 |
| h009-12 | 2.24E+06 | 9.17E−04 | 4.09E−10 |
| h009-13 | 2.28E+06 | 1.06E−03 | 4.65E−10 |
| h009-14 | 2.27E+06 | 1.85E−03 | 8.15E−10 |
| h009-15 | 2.28E+06 | 1.70E−03 | 7.45E−10 |
| ch-011 | 7.46E+06 | 1.48E−03 | 1.99E−10 |
| h011-01 | 6.68E+06 | 2.08E−03 | 3.11E−10 |
| h011-02 | 6.59E+06 | 1.95E−03 | 2.97E−10 |
| h011-03 | 6.97E+06 | 2.13E−03 | 3.06E−10 |
| h011-04 | 6.76E+06 | 2.02E−03 | 2.99E−10 |
| h011-05 | 6.89E+06 | 2.13E−03 | 3.09E−10 |
| h011-06 | 6.71E+06 | 2.01E−03 | 3.00E−10 |
| ch-160 | 2.74E+06 | 1.70E−04 | 6.20E−11 |
| h160-01 | 1.99E+06 | 1.36E−04 | 6.87E−11 |
| h160-02 | 1.93E+06 | 1.63E−04 | 8.46E−11 |
| h160-03 | 2.40E+06 | 1.87E−04 | 7.79E−11 |
| h160-04 | 2.12E+06 | E84E−04 | 8.67E−11 |
| h160-05 | 2.05E+06 | E41E−04 | 6.91E−11 |
| h160-06 | 2.09E+06 | E64E−04 | 7.82E−11 |
| h160-07 | 2.43E+06 | E82E−04 | 7.48E−11 |
| h160-08 | 2.29E+06 | E79E−04 | 7.82E−11 |

The results show that all the humanized antibodies obtained in the present disclosure have high affinity to human CD38.

TABLE 18
Results of the affinity of anti-CD38 antibody to
human CD38 by BIAcore

| Ligand | Analyte | ka (1/Ms) | kd (1/s) | KD (M) |
|---|---|---|---|---|
| Dara | Human CD38-His | 6.45E+05 | 1.51E−03 | 2.35E−09 |
| hu9E | | 1.05E+06 | 1.38E−03 | 1.31E−09 |
| hu11E | | 2.07E+06 | 1.17E−03 | 5.68E−10 |
| hu160E | | 1.96E+06 | 1.15E−04 | 5.85E−11 |

The results show that antibodies hu9E, hu11E and hu160E obtained in the present disclosure all have high affinity to human CD38, and the KD values are lower than that of the control antibody Dara, better than that of the control antibody.

In Vivo Evaluation of Biological Activity

### Test Example 8. In Vivo Pharmacokinetic Test of CD38 Antibodies

18 SD rats, male, were divided into 6 groups equally. The animals were provided by Sipur-Bikai Laboratory Animal Co., Ltd.; The animals were respectively administered by intravenous injection or subcutaneous injection, at a dosage of 3 mg/kg. For the group administered by intravenous injection, 0.2 ml of whole blood was collected without anticoagulation before dosing, 5 min, 8 h, 1 d, 2 d, 4 d, 7 d, 10 d, 14 d, 21 d and 28 d after administration; After collection, the blood was placed at 4° C. for 30 min, centrifuged at 1000 g for 15 min, and the supernatant (serum) was transferred into EP tubes and stored at −80° C.; For the group administered by subcutaneous injection, whole blood was collected before dosing, 1 h, 8 h, 1 d, 2 d, 4 d, 7 d, 10 d, 14 d, 21 d and 28 d after administration. The whole blood was collected on days 7, 10, 14, 21, and 28. The serum was isolated, transferred in EP tubes and store at −80° C.

Standard curves for the different samples were generated according to the method described in Test Example 1 (CD38 protein-binding ELISA of the anti-CD38 antibodies). The serum samples, in replacement of the anti-CD38 antibodies at 1:1000 dilution, were added into the the reaction system. The serum concentrations of the anti-CD38 antibodies at different time points were calculated based on OD450, and pharmacokinetic parameters were analyzed and calculated on the basis of the collected data by Phoenix WinNonlin software. The in vivo pharmacokinetic results of antibodies hu9E, hu11E and hu160E are shown in Table 19.

TABLE 19
Evaluation of pharmacokinetics in rat for antibodies

| | hu9E | | hu11E | | hu160E | |
|---|---|---|---|---|---|---|
| | IV | SC | IV | SC | IV | SC |
| dosage | 3 mg/kg | 3 mg/kg | 3 mg/kg | 3 mg/kg | 3 mg/kg | 3 mg/kg |
| Bioavailability | — | 81.4% | — | 113.8% | — | 113.9% |
| T1/2 (day) | 11.4 | 11.1 | 13.3 | 12.1 | 13.6 | 10.9 |

Note:
In the table, T1/2 means half-life, IV means intravenous injection, SC means subcutaneous injection.

PK (Pharmacokinetics) of hu9E, hu11E, and hu160E was measured in rats after subcutaneous and intravenous injection at a dosage of 3 mpk. The results show that the antibodies have favorable PK performance in rats: high bioavailability is observed in all antibodies when injected subcutaneously, and with the average T1/2 of intravenous injection is 11.4 days, 13.3 days and 13.6 days, respectively; the average T1/2 of subcutaneous injection is 11.1 days, 12.1 days and 10.9 days, respectively, suggesting favorable stability of antibodies in rats and the possibility to develop subcutaneous formulations.

### Test Example 9. In Vivo pharmacodynamics Test of CD38 antibodies on Tumor In Mice

Balb/c nude mice, SPF, 14-16 g, female, were purchased from Shanghai SLAC Laboratory Animal Co., Ltd. Balb/c nude mice were allowed to adapt for laboratory environment for 6 days, and were subcutaneously inoculated with AMO-1 cells (Cobioer, Nanjing, CBP60242, 5×10⁶+50% matrigel/mouse, basement membrane Matrigel, BD, Cat. No. #356237) into the right ribs. 9 days later, the mice were divided into a total of 7 groups, 8 mice/group, with average tumor volume of about 197.21±9.25 mm³ (d0). The groups were as follows:

Blank control group, IgG (3 mg/kg) (C25-hIgG1 (WT), laboratory-made);

Dara (1 mg/kg) group;

Dara (3 mg/kg) group;

hu11E (1 mg/kg) group;

hu11E (3 mg/kg) group;

hu160E (1 mg/kg) group;

hu160E (3 mg/kg) group;

Intraperitoneal injection was performed, twice a week, for 3 weeks. The tumor volume and body weight were measured twice a week, and the data were recorded. The mice were sacrificed and the tumors were removed after all the administrations were completed.

Excel 2003 statistical software was used to calculate the average (avg), SD value (STDEV) and SEM value (STDEV/SQRT); the P value for the difference between groups was calculated by TTEST.

The tumor volume (V) was calculated according to the following formula:

V=1/2×L_{long}Lₛₕₒᵣₜ²

Relative volume (RTV)=V_{T}/V₀

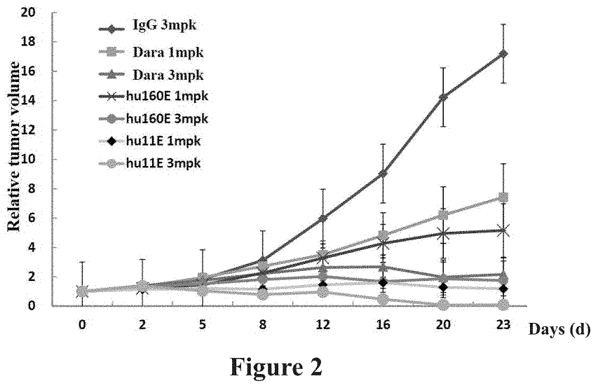

Tumor-inhibition rate (%)=(C_{RTV}−T_{RTV})/C_{RTV} (%)

wherein V₀ and V_{T} refer to the tumor volumes at the beginning and at the end of the experiment, respectively. C_{RTV} and T_{RTV} refer to the relative tumor volumes of the control group and the test group at the end of the experiment, respectively. The results are shown in Table 20 and FIG. 2.

TABLE 20
In vivo anti-tumor results of anti-CD38 antibodies

| Antibody | dosage | Tumor-inhibition rate (%) |
|---|---|---|
| Dara | 1 mpk | 56.83 |
| | 3 mpk | 87.44 |
| hu11E | 1 mpk | 93.14 |
| | 3 mpk | 99.52 |
| hu160E | 1 mpk | 70.02 |
| | 3 mpk | 89.89 |

Note:
mpk means mg/kg.

The results of in vivo anti-tumor efficiency in mice show that both the humanized antibodies hu11E and hu160E of the present disclosure can significantly inhibit the growth of tumor. Compared to the blank control IgG (3 mg/kg) group, hu11E (1 mg/kg) group and hu160E (1 mg/kg) group exhibit tumor-inhibition rate of 93.14% and 70.02%, respectively; hu11E (3 mg/kg) group and hu160E (3 mg/kg) group exhibit tumor-inhibition rate of 99.52% and 89.89%, respectively. During the administration process, the animals in each group displayed normal body weight, indicating that the antibodies of the present disclosure do not have obvious toxic and side effects.

1. An anti-CD38 antibody or an antigen-binding fragment thereof, wherein the anti-CD38 antibody or the antigen-binding fragment specifically binds to human CD38, the antibody or the antigen-binding fragment thereof comprising:
(i)
a heavy chain HCDR1, the amino acid sequence thereof is as shown in SEQ ID No: 15 or has 3, 2 or 1 amino acid(s) difference(s) when compared with SEQ ID No: 15,
a heavy chain HCDR2, the amino acid sequence thereof is as shown in SEQ ID No: 16 or has 3, 2 or 1 amino acid(s) difference(s) when compared with SEQ ID No: 16,
a heavy chain HCDR3, the amino acid sequence thereof is as shown in SEQ ID No: 17 or has 3, 2 or 1 amino acid(s) difference(s) when compared with SEQ ID No: 17,
a light chain LCDR1, the amino acid sequence thereof is as shown in SEQ ID No: 18 or has 3, 2 or 1 amino acid(s) difference(s) when compared with SEQ ID No: 18,
a light chain LCDR2, the amino acid sequence thereof is as shown in SEQ ID No: 19 or has 3, 2 or 1 amino acid(s) difference(s) when compared with SEQ ID No: 19, and
a light chain LCDR3, the amino acid sequence thereof is as shown in SEQ ID No: 20 or has 3, 2 or 1 amino acid(s) difference(s) when compared with SEQ ID No: 20; or
(ii)
a heavy chain HCDR1, the amino acid sequence thereof is as shown in SEQ ID No: 9 or has 3, 2 or 1 amino acid(s) difference(s) when compared with SEQ ID No: 9,
a heavy chain HCDR2, the amino acid sequence thereof is as shown in SEQ ID No: 10 or has 3, 2 or 1 amino acid(s) difference(s) when compared with SEQ ID No: 10,
a heavy chain HCDR3, the amino acid sequence thereof is as shown in SEQ ID No: 11 or has 3, 2 or 1 amino acid(s) difference(s) when compared with SEQ ID No: 11,
a light chain LCDR1, the amino acid sequence thereof is as shown in SEQ ID No: 12 or has 3, 2 or 1 amino acid(s) difference(s) when compared with SEQ ID No: 12,
a light chain LCDR2, the amino acid sequence thereof is as shown in SEQ ID No: 13 or has 3, 2 or 1 amino acid(s) difference(s) when compared with SEQ ID No: 13, and
a light chain LCDR3, the amino acid sequence thereof is as shown in SEQ ID No: 14 or has 3, 2 or 1 amino acid(s) difference(s) when compared with SEQ ID No: 14; or
(iii)
a heavy chain HCDR1, the amino acid sequence thereof is as shown in SEQ ID No: 15 or has 3, 2 or 1 amino acid(s) difference(s) when compared with SEQ ID No: 15,
a heavy chain HCDR2, the amino acid sequence thereof is as shown in SEQ ID No: 21 or has 3, 2 or 1 amino acid(s) difference(s) when compared with SEQ ID No: 21,
a heavy chain HCDR3, the amino acid sequence thereof is as shown in SEQ ID No: 17 or has 3, 2 or 1 amino acid(s) difference(s) when compared with SEQ ID No: 17,
a light chain LCDR1, the amino acid sequence thereof is as shown in SEQ ID No: 22 or has 3, 2 or 1 amino acid(s) difference(s) when compared with SEQ ID No: 22,
a light chain LCDR2, the amino acid sequence thereof is as shown in SEQ ID No: 19 or has 3, 2 or 1 amino acid(s) difference(s) when compared with SEQ ID No: 19, and
a light chain LCDR3, the amino acid sequence thereof is as shown in SEQ ID No: 23 or has 3, 2 or 1 amino acid(s) difference(s) when compared with SEQ ID No: 23.

2. The anti-CD38 antibody or the antigen-binding fragment thereof according to claim 1, wherein the antibody is murine antibody, chimeric antibody or humanized antibody.

3. The anti-CD38 antibody or the antigen-binding fragment thereof according to claim 2, wherein the murine antibody or chimeric antibody comprises a heavy chain variable region and a light chain variable region, wherein:
(a) the amino acid sequence of the heavy chain variable region is as shown in SEQ ID No: 3 or has at least 95% sequence identity to SEQ ID No: 3, and the amino acid sequence of the light chain variable region is as shown in SEQ ID No: 4 or has at least 95% sequence identity to SEQ ID No: 4;
(b) the amino acid sequence of the heavy chain variable region is as shown in SEQ ID No: 5 or has at least 95% sequence identity to SEQ ID No: 5, and the amino acid sequence of the light chain variable region is as shown in SEQ ID No: 6 or has at least 95% sequence identity to SEQ ID No: 6; or
(c) the amino acid sequence of the heavy chain variable region is as shown in SEQ ID No: 7 or has at least 95% sequence identity to SEQ ID No: 7, and the amino acid sequence of the light chain variable region is as shown in SEQ ID No: 8 or has at least 95% sequence identity to SEQ ID No: 8.

4. The anti-CD38 antibody or the antigen-binding fragment thereof according to claim 2, wherein the antibody is humanized antibody comprising framework regions or framework region variants derived from human antibody, and the framework region variants have up to 10 amino acid back-mutation(s) on light chain framework regions and/or heavy chain framework regions of the human antibody, respectively.

5. The anti-CD38 antibody or the antigen-binding fragment thereof according to claim 4, wherein the humanized antibody comprises: a heavy chain variable region as shown in SEQ ID Nos: 24, 32, or 37, or a variant thereof, wherein the variant has 1-10 amino acid mutation(s) on the framework regions of the heavy chain variable region as shown in SEQ ID Nos: 24, 32 or 37.

6. The anti-CD38 antibody or the antigen-binding fragment thereof according to claim 5, wherein the variant is selected from any one of the following (g) to (i):
(g) one or more back-mutations selected from the group consisting of 2F, 38K, 44S, 48I, 67A, 66K, 69L, 71V and 73Q on the framework regions of the heavy chain variable region as shown in SEQ ID No: 24;
(h) one or more back-mutations selected from the group consisting of 79F and 91S on the framework regions of the heavy chain variable region as shown in SEQ ID No: 32; and
(i) back-mutation of 48I on the framework regions of the heavy chain variable region as shown in SEQ ID No: 37.

7. The anti-CD38 antibody or the antigen-binding fragment thereof according to claim 4, wherein the humanized antibody comprises a light chain variable region as shown in SEQ ID Nos: 25, 33 or 38 or a variant thereof, wherein the variant has 1-10 amino acid mutation(s) on the framework regions of the light chain variable region as shown in SEQ ID Nos: 25, 33 or 38.

8. The anti-CD38 antibody or the antigen-binding fragment thereof according to claim 7, wherein the variant is selected from any one of the following (j) to (l):
(j) one or more back-mutations selected from the group consisting of 2F, 43S, 49K and 87F on the framework regions of the light chain variable region as shown in SEQ ID No: 25;
(k) one or more back-mutations selected from the group consisting of 58I, 68R and 85T on the framework regions of the light chain variable region as shown in SEQ ID No: 33;
(l) one or more back-mutations selected from the group consisting of 4L, 9A, 22S, 58I, 60A and 68R on the framework regions of the light chain variable region as shown in SEQ ID No: 38.

9. The anti-CD38 antibody or the antigen-binding fragment thereof according to claim 1, comprising:
(m) a heavy chain variable region as shown in SEQ ID Nos: 24, 26, 27, 28 or 29, and a light chain variable region as shown in SEQ ID Nos: 25, 30 or 31;
(n) a heavy chain variable region as shown in SEQ ID Nos: 32 or 34, and a light chain variable region as shown in SEQ ID No:33, 35 or 36; or
(o) a heavy chain variable region as shown in SEQ ID Nos: 37 or 39, and a light chain variable region as shown in SEQ ID No:38, 40, 41 or 42.

10. The anti-CD38 antibody or the antigen-binding fragment thereof according to claim 1, wherein the antibody comprises constant regions.

11. The anti-CD38 antibody or the antigen-binding fragment thereof according to claim 10, comprising:
a heavy chain as shown in the amino acid sequence of SEQ ID Nos: 46, 48, 49, 51, 52 or 54 or having at least 85% sequence identity to the amino acid sequence of SEQ ID Nos: 46, 48, 49, 51, 52 or 54; and/or
a light chain as shown in the amino acid sequence of SEQ ID Nos: 47, 50 or 53, or having at least 85% sequence identity to the amino acid sequence of SEQ ID Nos: 47, 50 or 53.

12. The anti-CD38 antibody or the antigen-binding fragment thereof according to claim 11, wherein the antibody comprises:
a heavy chain as shown in SEQ ID No: 46 and a light chain as shown in SEQ ID No: 47;
a heavy chain as shown in SEQ ID No: 48 and a light chain as shown in SEQ ID No: 47;
a heavy chain as shown in SEQ ID No: 49 and a light chain as shown in SEQ ID No: 50;
a heavy chain as shown in SEQ ID No: 51 and a light chain as shown in SEQ ID No: 50;
a heavy chain as shown in SEQ ID No: 52 and a light chain as shown in SEQ ID No: 53; or
a heavy chain as shown in SEQ ID No: 54 and a light chain as shown in SEQ ID No: 53.

13. (canceled)

14. A pharmaceutical composition comprising:
a therapeutically effective amount of the anti-CD38 antibody or the antigen-binding fragment thereof according to claim 1, and
one or more pharmaceutically acceptable carriers, diluents, buffers or excipients.

15. An isolated nucleic acid molecule encoding the anti-CD38 antibody or the antigen-binding fragment thereof according to claim 1.

16. (canceled)

17. (canceled)

18. A method for preparing the anti-CD38 antibody or the antigen-binding fragment thereof according to claim 1, the method comprising:
cultivating a host cell,
recovering the anti-CD38 antibody or the antigen-binding fragment thereof;
optionally, purifying the anti-CD38 antibody or the antigen-binding fragment thereof.

19. A method for detecting or measuring human CD38, comprising:
contacting the anti-CD38 antibody or the antigen-binding fragment thereof according to claim 1 with a sample to be tested;
determining the presence or level of human CD38 in the sample to be tested.

20. (canceled)

21. A method of treating or preventing a disease or a disorder in a subject in need thereof, the method comprising administering to the subject a therapeutically effective amount or a prophylactically effective amount of the anti-CD38 antibody or the antigen-binding fragment thereof according to claim 1.

22. The method according to claim 21, wherein the disease or disorder is CD38 positive disease or disorder.

23. The method according to claim 21, wherein
the disease or disorder is tumor or immune disease;
wherein the immune disease is selected from the group consisting of: rheumatoid arthritis, psoriasis, ankylosing spondylitis, joint psoriasis, dermatitis, systemic scleroderma and sclerosis, inflammatory bowel disease, Crohn's disease, ulcerative colitis, respiratory distress syndrome, meningitis, encephalitis, gastritis, uveitis, glomerulonephritis, eczema, asthma, arteriosclerosis, leukocyte adhesion deficiency, Raynaud syndrome, Sjogren syndrome, juvenile diabetes, Reiter disease, Behcet disease, immune complex nephritis, IgA nephropathy, IgM polyneuropathy, immune-mediated thrombocytopenia symptom, hemolytic anemia, myasthenia gravis, lupus nephritis, systemic lupus erythematosus, rheumatoid arthritis, atopic dermatitis, pemphigus, Graves disease, Hashimoto's thyroiditis, Wegener's granulomatosis, Omenn syndrome, chronic renal failure, acute infectious mononucleosis, multiple sclerosis, HIV and herpes virus-related diseases, severe acute respiratory syndrome and chorioretinitis, graft versus host disease, and immune disease caused by virus infection; and
wherein the tumor is selected from the group consisting of: leukemia, B cell lymphoma, T cell lymphoma, NK cell lymphoma, plasma cell malignant tumor and myeloma, the B cell lymphoma is selected from the group consisting of: mature B cell tumor, precursor B cell lymphoblastic leukemia/lymphoma, B cell non-Hodgkin's lymphoma and B cell Hodgkin's lymphoma, acute lymphocytic leukemia, acute lymphoblastic leukemia, acute promyelocytic leukemia, chronic lymphocytic leukemia, acute or chronic myeloid leukemia, multiple myeloma, anterior medullary tumor, light chain amyloidosis, B cell chronic lymphocytic leukemia, small lymphocytic leukemia, B cell acute lymphocytic leukemia, B cell prelymphocytic leukemia, lymphoplasmacytoid lymphoma, mantle cell lymphoma, follicular lymphoma, cutaneous follicular central lymphoma, marginal zone B cell lymphoma, hairy cell leukemia, diffuse large B cell lymphoma, Burkitt lymphoma, plasma cell tumor, plasma cell myeloma, plasma cell leukemia, post-transplantation lymphoproliferative diseases, Waldenstrom macroglobulinemia, plasma cell leukemia and anaplastic large cell lymphoma and hairy cell lymphoma.

24. The anti-CD38 antibody or the antigen-binding fragment thereof according to claim 4, wherein the humanized antibody comprises any one selected from the following (d) to (f):
(d) a heavy chain variable region, wherein the heavy chain variable region comprising: heavy chain HCDR1, HCDR2, HCDR3 and heavy chain framework region(s), wherein the amino acid sequence of the HCDR1 is as shown in SEQ ID No: 9 or has 3, 2 or 1 amino acid(s) difference(s) when compared with SEQ ID No: 9, the amino acid sequence of the HCDR2 is as shown in SEQ ID No: 10 or has 3, 2 or 1 amino acid(s) difference(s) when compared with SEQ ID No: 10, the amino acid of the HCDR3 is as shown in SEQ ID No: 11 or has 3, 2 or 1 amino acid(s) difference(s) when compared with SEQ ID No: 11, and the heavy chain framework region(s) comprise(s) one or more back-mutation(s) selected from the group consisting of: 2F, 38K, 44S, 48I, 67A, 66K, 69L, 71V and 73Q; and/or
a light chain variable region, wherein the light chain variable region comprising: light chain LCDR1, LCDR2, LCDR3 and light chain framework region(s), wherein the amino acid sequence of the LCDR1 is as shown in SEQ ID No: 12 or has 3, 2 or 1 amino acid(s) difference(s) when compared with SEQ ID No: 12, the amino acid sequence of the LCDR2 is as shown in SEQ ID No: 13 or has 3, 2 or 1 amino acid(s) difference(s) when compared with SEQ ID No: 13, the amino acid of the LCDR3 is as shown in SEQ ID No: 14 or has 3, 2 or 1 amino acid(s) difference(s) when compared with SEQ ID No: 14, and the light chain framework region(s) comprise(s) one or more back-mutation(s) selected from the group consisting of: 2F, 43S, 49K and 87F;
(e) a heavy chain variable region, wherein the heavy chain variable region comprising: heavy chain HCDR1, HCDR2, HCDR3 and heavy chain framework region(s), wherein the amino acid sequence of the HCDR1 is as shown in SEQ ID No: 15 or has 3, 2 or 1 amino acid(s) difference(s) when compared with SEQ ID No: 15, the amino acid sequence of the HCDR2 is as shown in SEQ ID No: 16 or has 3, 2 or 1 amino acid(s) difference(s) when compared with SEQ ID No: 16, the amino acid of the HCDR3 is as shown in SEQ ID No: 17 or has 3, 2 or 1 amino acid(s) difference(s) when compared with SEQ ID No: 17, and the heavy chain framework region(s) comprise(s) one or more back-mutation(s) selected from the group consisting of: 79F, 82A T, 91S and 76S; and/or
a light chain variable region, wherein the light chain variable region comprising: light chain LCDR1, LCDR2, LCDR3 and light chain framework region(s), wherein the amino acid sequence of the LCDR1 is as shown in SEQ ID No: 18 or has 3, 2 or 1 amino acid(s) difference(s) when compared with SEQ ID No: 18, the amino acid sequence of the LCDR2 is as shown in SEQ ID No: 19 or has 3, 2 or 1 amino acid(s) difference(s) when compared with SEQ ID No: 19, the amino acid of the LCDR3 is as shown in SEQ ID No: 20 or has 3, 2 or 1 amino acid(s) difference(s) when compared with SEQ ID No: 20, and the light chain framework region(s) comprise(s) one or more back-mutation(s) selected from the group consisting of: 58I, 68R and 85T; and
(f) a heavy chain variable region, wherein the heavy chain variable region comprising: heavy chain HCDR1, HCDR2, HCDR3 and heavy chain framework region(s), wherein the amino acid sequence of the HCDR1 is as shown in SEQ ID No: 15 or has 3, 2 or 1 amino acid(s) difference(s) when compared with SEQ ID No: 15, the amino acid sequence of the HCDR2 is as shown in SEQ ID No: 21 or has 3, 2 or 1 amino acid(s) difference(s) when compared with SEQ ID No: 21, the amino acid of the HCDR3 is as shown in SEQ ID No: 17 or has 3, 2 or 1 amino acid(s) difference(s) when compared with SEQ ID No: 17, and the heavy chain framework region(s) comprise(s) one or more back-mutation(s) selected from the group consisting of: 48I, 77T and 82A T; and/or
a light chain variable region, wherein the light chain variable region comprising: light chain LCDR1, LCDR2, LCDR3 and light chain framework region(s), wherein the amino acid sequence of the LCDR1 is as shown in SEQ ID No: 22 or has 3, 2 or 1 amino acid(s) difference(s) when compared with SEQ ID No: 22, the amino acid sequence of the LCDR2 is as shown in SEQ ID No: 19 or has 3, 2 or 1 amino acid(s) difference(s) when compared with SEQ ID No: 19, the amino acid of the LCDR3 is as shown in SEQ ID No: 23 or has 3, 2 or 1 amino acid(s) difference(s) when compared with SEQ ID No: 23, and the light chain framework region(s) comprise(s) one or more back-mutation(s) selected from the group consisting of: 4L, 9A, 22S, 58I, 60A and 68R;
wherein the back-mutation sites are numbered according to Kabat numbering criteria.

25. The anti-CD38 antibody or the antigen-binding fragment thereof according to claim 6, wherein the humanized antibody comprises:
a heavy chain variable region as shown in SEQ ID Nos: 26, 27, 28, 29, 34 or 39, or
a heavy chain variable region having at least 95% sequence identity to SEQ ID Nos: 26, 27, 28, 29, 34, or 39.

26. The anti-CD38 antibody or the antigen-binding fragment thereof according to claim 8, wherein the humanized antibody comprises:
a light chain variable region as shown in SEQ ID Nos: 30, 31, 35, 36, 40, 41 or 42, or
a light chain variable region having at least 95% sequence identity to SEQ ID Nos: 30, 31, 35, 36, 40, 41 or 42.

27. The anti-CD38 antibody or the antigen-binding fragment thereof according to claim 9, wherein the humanized antibody comprises:
(p) a heavy chain variable region as shown in SEQ ID No: 26 and a light chain variable region as shown in sequence SEQ ID No: 30; or
(q) a heavy chain variable region as shown in SEQ ID No: 32 and a light chain variable region as shown in SEQ ID No: 33; or
(r) a heavy chain variable region as shown in SEQ ID No: 37 and a light chain variable region as shown in SEQ ID No: 38.


SEQUENCE LISTING (57 sequences)

SEQ ID NO: 1 - protein, 491 aa, Artificial Sequence
  PEPTIDE: Fusion protein of CD38 extracellular domain and mouse IgG2a-FcCD38-ECD-mFc
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 490 aa, Artificial Sequence
  PEPTIDE: Fusion protein of CD38 extracellular domain and human IgG1Fc
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 118 aa, Mus musculus
  DOMAIN: m009 heavy chain variable region sequence
EFQLQQSGPELVKPGASVKISCKASGYSFTDYNLNWVKQSNGKSLEWIGVINPKYDAINYNQKFKDKATLTVDQSSSTAYMQLSSLTSEDSAVYYCAREGWGKALDYWGPGTSVIVSS

SEQ ID NO: 4 - protein, 107 aa, Mus musculus
  DOMAIN: m009 light chain variable region sequence
DFVLTQSPATLSVTPGDSVSLSCRASQSIYTNLHWYQQKSHESPRLLIKYASQSISGIPSRFSGSGSGTDFTLSINSVETEDSGMYFCQQSNSWPLTFGAGTKLELK

SEQ ID NO: 5 - protein, 120 aa, Mus musculus
  DOMAIN: m011 heavy chain variable region sequence
EVQLVESGGGLVKPGGSLKLSCAASGFTFSDYGMHWVRQAPEKGLEWVAFISSGSSSIYYADTVKGRFTISRDNAKNTLFLQMTSLRSEDTAMYSCARNYVSSYGYFDYWGQGTTLTVSS

SEQ ID NO: 6 - protein, 111 aa, Mus musculus
  DOMAIN: m011 light chain variable region sequence
DIVMTQSPASLAVSLGQRATISCRASENVDNYGISFMHWYQQKPGQPPKLLIYRASNLESGIPARFSGSGSRTDFTLTINPVETDDVATYYCQQSNKDPLTFGSGTKLEIK

SEQ ID NO: 7 - protein, 120 aa, Mus musculus
  DOMAIN: m160 heavy chain variable region sequence
EVQLVESGGGLVKPGGSLKLSCVASGFTFSDYGMHWVRQAPEKGLEWIAFISTGSSNIYYVDKVKGRFTISRDNAKNTLFLQMTSLRSEDTAMYYCARNYVSSYGYFDYWGQGTTLTVSS

SEQ ID NO: 8 - protein, 111 aa, Mus musculus
  DOMAIN: m160 light chain variable region sequence
DIVLTQSPASLAVSLGQRATVSCRASESVDNYGISFMHWYQQKPGQPPKLLIYRASNLESGIPARFSGSGSRTDFTLTINPVETDDVATYYCQQTNKDPLTFGGGTKLELK

SEQ ID NO: 9 - protein, 5 aa, Mus musculus
  DOMAIN: m009 HCDR1
DYNLN

SEQ ID NO: 10 - protein, 17 aa, Mus musculus
  DOMAIN: m009 HCDR2
VINPKYDAINYNQKFKD

SEQ ID NO: 11 - protein, 9 aa, Mus musculus
  DOMAIN: m009 HCDR3
EGWGKALDY

SEQ ID NO: 12 - protein, 11 aa, Mus musculus
  DOMAIN: m009 LCDR1
RASQSIYTNLH

SEQ ID NO: 13 - protein, 7 aa, Mus musculus
  DOMAIN: m009 LCDR2
YASQSIS

SEQ ID NO: 14 - protein, 9 aa, Mus musculus
  DOMAIN: m009 LCDR3
QQSNSWPLT

SEQ ID NO: 15 - protein, 5 aa, Mus musculus
  DOMAIN: m011 HCDR1
DYGMH

SEQ ID NO: 16 - protein, 17 aa, Mus musculus
  DOMAIN: m011 HCDR2
FISSGSSSIYYADTVKG

SEQ ID NO: 17 - protein, 11 aa, Mus musculus
  DOMAIN: m011 HCDR3
NYVSSYGYFDY

SEQ ID NO: 18 - protein, 15 aa, Mus musculus
  DOMAIN: m011 LCDR1
RASENVDNYGISFMH

SEQ ID NO: 19 - protein, 7 aa, Mus musculus
  DOMAIN: m011 LCDR2
RASNLES

SEQ ID NO: 20 - protein, 9 aa, Mus musculus
  DOMAIN: m011 LCDR3
QQSNKDPLT

SEQ ID NO: 21 - protein, 17 aa, Mus musculus
  DOMAIN: m160 HCDR2
FISTGSSNIYYVDKVKG

SEQ ID NO: 22 - protein, 15 aa, Mus musculus
  DOMAIN: m160 LCDR1
RASESVDNYGISFMH

SEQ ID NO: 23 - protein, 9 aa, Mus musculus
  DOMAIN: m160 LCDR3
QQTNKDPLT

SEQ ID NO: 24 - protein, 118 aa, Artificial Sequence
  DOMAIN: h009 VH1
EVQLVQSGAEVKKPGASVKVSCKASGYTFTDYNLNWVRQAPGQRLEWMGVINPKYDAINYNQKFKDRVTITRDTSASTAYMELSSLRSEDTAVYYCAREGWGKALDYWGQGTLVTVSS

SEQ ID NO: 25 - protein, 107 aa, Artificial Sequence
  DOMAIN: h009 VL1
EIVLTQSPATLSLSPGERATLSCRASQSIYTNLHWYQQKPGQAPRLLIYYASQSISGIPARFSGSGSGTDFTLTISSLEPEDFAVYYCQQSNSWPLTFGGGTKVEIK

SEQ ID NO: 26 - protein, 118 aa, Artificial Sequence
  DOMAIN: h009 VH2
EFQLVQSGAEVKKPGASVKVSCKASGYTFTDYNLNWVRQAPGQSLEWMGVINPKYDAINYNQKFKDRVTITVDTSASTAYMELSSLRSEDTAVYYCAREGWGKALDYWGQGTLVTVSS

SEQ ID NO: 27 - protein, 118 aa, Artificial Sequence
  DOMAIN: h009 VH3
EFQLVQSGAEVKKPGASVKVSCKASGYTFTDYNLNWVRQAPGQSLEWIGVINPKYDAINYNQKFKDRATITVDTSASTAYMELSSLRSEDTAVYYCAREGWGKALDYWGQGTLVTVSS

SEQ ID NO: 28 - protein, 118 aa, Artificial Sequence
  DOMAIN: h009 VH4
EFQLVQSGAEVKKPGASVKVSCKASGYTFTDYNLNWVKQAPGQSLEWMGVINPKYDAINYNQKFKDKVTLTVDQSASTAYMELSSLRSEDTAVYYCAREGWGKALDYWGQGTLVTVSS

SEQ ID NO: 29 - protein, 118 aa, Artificial Sequence
  DOMAIN: h009 VH5
EFQLVQSGAEVKKPGASVKVSCKASGYTFTDYNLNWVKQAPGQSLEWIGVINPKYDAINYNQKFKDKATLTVDQSASTAYMELSSLRSEDTAVYYCAREGWGKALDYWGQGTLVTVSS

SEQ ID NO: 30 - protein, 107 aa, Artificial Sequence
  DOMAIN: h009 VL2
EIVLTQSPATLSLSPGERATLSCRASQSIYTNLHWYQQKPGQSPRLLIKYASQSISGIPARFSGSGSGTDFTLTISSLEPEDFAVYYCQQSNSWPLTFGGGTKVEIK

SEQ ID NO: 31 - protein, 107 aa, Artificial Sequence
  DOMAIN: h009 VL3
EFVLTQSPATLSLSPGERATLSCRASQSIYTNLHWYQQKPGQSPRLLIKYASQSISGIPARFSGSGSGTDFTLTISSLEPEDFAVYFCQQSNSWPLTFGGGTKVEIK

SEQ ID NO: 32 - protein, 120 aa, Artificial Sequence
  DOMAIN: h011 VH1
EVQLVESGGGLVQPGGSLRLSCAASGFTFSDYGMHWVRQAPGKGLEWVAFISSGSSSIYYADTVKGRFTISRDNAKSSLYLQMTSLRAEDTAVYYCARNYVSSYGYFDYWGQGTTVTVSS

SEQ ID NO: 33 - protein, 111 aa, Artificial Sequence
  DOMAIN: h011 VL1
DIVMTQSPDSLAVSLGERATINCRASENVDNYGISFMHWYQQKPGQPPKLLIYRASNLESGVPDRFSGSGSGTDFTLTISSLQAEDVAVYYCQQSNKDPLTFGGGTKVEIK

SEQ ID NO: 34 - protein, 120 aa, Artificial Sequence
  DOMAIN: h011 VH2
EVQLVESGGGLVQPGGSLRLSCAASGFTFSDYGMHWVRQAPGKGLEWVAFISSGSSSIYYADTVKGRFTISRDNAKSSLFLQMTSLRAEDTAVYSCARNYVSSYGYFDYWGQGTTVTVSS

SEQ ID NO: 35 - protein, 111 aa, Artificial Sequence
  DOMAIN: h011 VL2
DIVMTQSPDSLAVSLGERATINCRASENVDNYGISFMHWYQQKPGQPPKLLIYRASNLESGVPDRFSGSGSRTDFTLTISSLQAEDVAVYYCQQSNKDPLTFGGGTKVEIK

SEQ ID NO: 36 - protein, 111 aa, Artificial Sequence
  DOMAIN: h011VL3
DIVMTQSPDSLAVSLGERATINCRASENVDNYGISFMHWYQQKPGQPPKLLIYRASNLESGIPDRFSGSGSRTDFTLTISSLQAEDVATYYCQQSNKDPLTFGGGTKVEIK

SEQ ID NO: 37 - protein, 120 aa, Artificial Sequence
  DOMAIN: h160 VH1
EVQLVESGGGLVQPGGSLRLSCAASGFTFSDYGMHWVRQAPGKGLEWVAFISTGSSNIYYVDKVKGRFTISRDNAKNTLYLQMTSLRAEDTAVYYCARNYVSSYGYFDYWGQGTTVTVSS

SEQ ID NO: 38 - protein, 111 aa, Artificial Sequence
  DOMAIN: h160 VL1
DIVMTQSPASLAVSLGERATINCRASESVDNYGISFMHWYQQKPGQPPKLLIYRASNLESGVPDRFSGSGSGTDFTLTISSLQAEDVAVYYCQQTNKDPLTFGGGTKVEIK

SEQ ID NO: 39 - protein, 120 aa, Artificial Sequence
  DOMAIN: h160 VH2
EVQLVESGGGLVQPGGSLRLSCAASGFTFSDYGMHWVRQAPGKGLEWIAFISTGSSNIYYVDKVKGRFTISRDNAKNTLYLQMTSLRAEDTAVYYCARNYVSSYGYFDYWGQGTTVTVSS

SEQ ID NO: 40 - protein, 111 aa, Artificial Sequence
  DOMAIN: h160 VL2
DIVLTQSPASLAVSLGERATINCRASESVDNYGISFMHWYQQKPGQPPKLLIYRASNLESGVPDRFSGSGSGTDFTLTISSLQAEDVAVYYCQQTNKDPLTFGGGTKVEIK

SEQ ID NO: 41 - protein, 111 aa, Artificial Sequence
  DOMAIN: h160 VL3
DIVLTQSPASLAVSLGERATINCRASESVDNYGISFMHWYQQKPGQPPKLLIYRASNLESGVPARFSGSGSRTDFTLTISSLQAEDVAVYYCQQTNKDPLTFGGGTKVEIK

SEQ ID NO: 42 - protein, 111 aa, Artificial Sequence
  DOMAIN: h160 VL4
DIVLTQSPASLAVSLGERATISCRASESVDNYGISFMHWYQQKPGQPPKLLIYRASNLESGIPARFSGSGSRTDFTLTISSLQAEDVAVYYCQQTNKDPLTFGGGTKVEIK

SEQ ID NO: 43 - protein, 330 aa, Artificial Sequence [sequence omitted]

SEQ ID NO: 44 - protein, 330 aa, Artificial Sequence
  DOMAIN: IgG1-E333A heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 107 aa, Artificial Sequence [sequence omitted]

SEQ ID NO: 46 - protein, 448 aa, Artificial Sequence
  CHAIN: h009-07 antibody heavy chain
EFQLVQSGAEVKKPGASVKVSCKASGYTFTDYNLNWVRQAPGQSLEWMGVINPKYDAINYNQKFKDRVTITVDTSASTAYMELSSLRSEDTAVYYCAREGWGKALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, Artificial Sequence
  CHAIN: h009-07/hu9E antibody light chain
EIVLTQSPATLSLSPGERATLSCRASQSIYTNLHWYQQKPGQSPRLLIKYASQSISGIPARFSGSGSGTDFTLTISSLEPEDFAVYYCQQSNSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 448 aa, Artificial Sequence
  CHAIN: hu9E antibody heavy chain
EFQLVQSGAEVKKPGASVKVSCKASGYTFTDYNLNWVRQAPGQSLEWMGVINPKYDAINYNQKFKDRVTITVDTSASTAYMELSSLRSEDTAVYYCAREGWGKALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 450 aa, Artificial Sequence
  CHAIN: h011-01 antibody heavy chain
EVQLVESGGGLVQPGGSLRLSCAASGFTFSDYGMHWVRQAPGKGLEWVAFISSGSSSIYYADTVKGRFTISRDNAKSSLYLQMTSLRAEDTAVYYCARNYVSSYGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 218 aa, Artificial Sequence
  CHAIN: h011-01/hu11E antibody light chain
DIVMTQSPDSLAVSLGERATINCRASENVDNYGISFMHWYQQKPGQPPKLLIYRASNLESGVPDRFSGSGSGTDFTLTISSLQAEDVAVYYCQQSNKDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 450 aa, Artificial Sequence
  CHAIN: hu11E antibody heavy chain
EVQLVESGGGLVQPGGSLRLSCAASGFTFSDYGMHWVRQAPGKGLEWVAFISSGSSSIYYADTVKGRFTISRDNAKSSLYLQMTSLRAEDTAVYYCARNYVSSYGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 450 aa, Artificial Sequence
  CHAIN: h160-01 antibody heavy chain
EVQLVESGGGLVQPGGSLRLSCAASGFTFSDYGMHWVRQAPGKGLEWVAFISTGSSNIYYVDKVKGRFTISRDNAKNTLYLQMTSLRAEDTAVYYCARNYVSSYGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 218 aa, Artificial Sequence
  CHAIN: h160-01/hu160E antibody light chain
DIVMTQSPASLAVSLGERATINCRASESVDNYGISFMHWYQQKPGQPPKLLIYRASNLESGVPDRFSGSGSGTDFTLTISSLQAEDVAVYYCQQTNKDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 450 aa, Artificial Sequence
  CHAIN: hu160E antibody heavy chain
EVQLVESGGGLVQPGGSLRLSCAASGFTFSDYGMHWVRQAPGKGLEWVAFISTGSSNIYYVDKVKGRFTISRDNAKNTLYLQMTSLRAEDTAVYYCARNYVSSYGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 452 aa, Artificial Sequence
  CHAIN: Dara heavy chain sequence
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial Sequence
  CHAIN: Dara light chain sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 264 aa, Artificial Sequence
  PEPTIDE: Fusion protein of CD38 extracellular domain with His tag CD38-ECD-His
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIHHHHHH